(19) United States
(12) Patent Application Publication
(10) Pub. No.: US 2024/0404634 A1
(43) Pub. Date: Dec. 5, 2024

(54) RESIDUAL ARTIFICIAL NEURAL NETWORK TO GENERATE PROTEIN SEQUENCES

(71) Applicant: Just-Evotec Biologics, Inc., Seattle, WA (US)

(72) Inventors: Joshua Smith, Seattle, WA (US); Jeremy Martin Shaver, Lake Forest Park, WA (US); Tileli Amimeur, Seattle, WA (US); Randal Robert Ketchem, Shalimar, FL (US); John Alex Taylor, Bellevue, WA (US)

(21) Appl. No.: 18/688,263

(22) PCT Filed: Aug. 31, 2022
(86) PCT No.: PCT/US2022/075765

§ 371 Date: Feb. 29, 2024

Related U.S. Application Data
(60) Provisional application No. 63/239,321, filed on Aug. 31, 2021.

(51) Int. Cl. G16B 30/00; G16B 40/00

(52) U.S. Cl. CPC G16B 30/00; G16B 40/00

(57) ABSTRACT

Amino acid sequences of base proteins can be analyzed by a neural network component. Loss values can be determined by the neural network component for individual positions of the base proteins. Variant protein sequences can be generated based on the base protein sequences and the loss values.

### PRIORITY CLAIM AND INCORPORATION BY REFERENCE

This application claims priority to U.S. Provisional Patent Application No. 63/239,321 filed Aug. 31, 2021, and entitled “Residual Artificial Neural Network to Generate Protein Sequences,” the entire contents of which is incorporated by reference herein in its entirety.

### BACKGROUND

Proteins are biological molecules that are comprised of one or more chains of amino acids. Proteins can have various functions within an organism. For example, some proteins can be involved in causing a reaction to take place within an organism. In other examples, proteins can transport molecules throughout the organism. In still other examples, proteins can be involved in the replication of genes. Additionally, some proteins can have therapeutic properties and be used to treat various biological conditions. The structure and function of proteins are based on the arrangement of amino acids that comprise the proteins. The arrangement of amino acids for proteins can be represented by a sequence of letters with each letter corresponding to an amino acid at a respective position. The arrangement of amino acids for proteins can also be represented by three dimensional structures that not only indicate the amino acids at various locations of the protein, but also indicate three dimensional features of the proteins, such as an a-helix or a P-sheet.

### BRIEF DESCRIPTION OF THE DRAWINGS

The present disclosure is illustrated by way of example and not limitation in the figures of the accompanying drawings, in which like references indicate similar elements.

FIG. 1 is a diagram illustrating an example framework to generate protein sequences based on loss values for a base sequence determined by a neural network component, in accordance with one or more implementations.

FIG. 2 is a diagram illustrating an example framework to train a generative adversarial network using transfer learning techniques to generate training data for a protein sequence generating system that includes a neural network component, in accordance with one or more implementations.

FIG. 3 is a diagram illustrating an example framework to generate humanized protein sequences using human template protein sequences and supplemental non-human protein sequences, in accordance with one or more implementations.

FIG. 4 is a diagram illustrating an example framework to generate protein sequences directed to a respective germline using a neural network component, in accordance with one or more implementations.

FIG. 5 is a diagram illustrating an example framework to generate sequences of antibodies using a neural network component, in accordance with one or more implementations.

FIG. 6 is a diagram illustrating an example framework to generate protein sequences using a residual neural network, in accordance with one or more implementations.

FIG. 7 illustrates an example framework that includes a schema for arranging amino acids of antibodies and the use of different dilation rates to traverse an example amino acid sequence arranged according to the schema, in accordance with one or more implementations.

FIG. 8 is a flow diagram illustrating an example process to generate protein sequences using a neural network, in accordance with one or more implementations.

FIG. 9A illustrates training loss that takes place in response to training a neural network using existing techniques and FIG. 9B illustrates training loss that takes place in response to training a neural network in accordance with techniques described herein.

FIG. 10 includes a number of scatter plots indicating correlations between loss scores generated according to implementations described herein and experimental measurements for a set of antibodies.

FIG. 11 illustrates a diagrammatic representation of a machine in the form of a computer system within which a set of instructions may be executed for causing the machine to perform any one or more of the methodologies discussed herein, according to an example implementation.

### DETAILED DESCRIPTION

Proteins can have many beneficial uses within organisms. In particular situations, proteins can be used to treat diseases and other biological conditions that can detrimentally impact the health of humans and other mammals. In various scenarios, proteins can participate in reactions that are beneficial to subjects and that can counteract one or more biological conditions being experienced by the subjects. In some examples, proteins can also bind to target molecules within an organism that may be detrimental to the health of a subject. For these reasons, many individuals and organizations have sought to develop proteins that may have therapeutic benefits.

The development of proteins can be a time consuming and resource intensive process. Often, candidate proteins for development can be identified as potentially having various biophysical properties, structural features (e.g., negatively charged patches, hydrophobic patches), three-dimensional (3D) structures, and/or one or more functions within an organism. In order to determine whether the candidate proteins have the characteristics of interest, the proteins can be synthesized and then tested to determine whether the actual characteristics of the synthesized proteins correspond to the desired characteristics. Due to the amount of resources needed to synthesize and test proteins for specified biophysical properties, structural features, 3D structures, and/or functions, the number of candidate proteins synthesized for therapeutic purposes is limited. In some situations, the number of proteins synthesized for therapeutic purposes can be limited by the loss of resources that takes place when candidate proteins are synthesized and do not have the desired characteristics.

The techniques, methods, frameworks, and systems described herein can include training a neural network component using training data that includes protein sequences. In various examples, input can be provided to the neural network component that includes a base protein sequence. The base protein sequence can be analyzed by the neural network component to determine an amount of loss with respect to a respective amino acid located at individual positions of the base protein sequence. The amount of loss can indicate a difference between a probability of an actual amino acid located at a position of the base protein sequence and a probability of an expected amino acid associated with the position based on an analysis of the training data. In various examples, the amount of loss can indicate a difference in a value of a characteristic of the base protein in relation to an expected value of the characteristic for the base protein based on an analysis of the training data.

The amount of loss for amino acids at individual positions of a base protein sequence can be used to generate variant protein sequences. In various examples, in situations where an amount of loss at a given position of a base protein sequence is at least a threshold amount of loss, the amino acid at the given position of the base protein sequence can be replaced. A replacement amino acid for the given position of the base protein sequence can correspond to an amino acid that minimizes an amount of loss with respect to a variant protein sequence that corresponds to the base protein sequence. In one or more examples, the replacement amino acid for a given position of the base protein sequence can increase a value of characteristic for the variant protein in relation to the value of the characteristic of the base protein.

In one or more implementations, a neural network component can be trained to determine an amount of loss with respect to non-human base protein sequences and, based on the amount of loss, a portion of the amino acids of the base protein sequence can be replaced such that the variant protein sequence corresponds more to a human protein sequence. In this way, humanized protein sequences can be generated using systems, techniques, and systems described herein. Additionally, a neural network component can be trained to determine an amount of loss with respect to base protein sequences that correspond to a first germline and amino acids of one or more positions of the base protein sequence can be modified to generate variant protein sequences that correspond to a second germline.

In one or more examples, the neural network component can include a convolutional neural network. In one or more illustrative examples, the neural network component can include one or more one-dimensional convolutional layers. Additionally, the neural network component can include a plurality of residual components. Output from an individual residual component can be fed into an additional residual component. In one or more examples, a dilation rate for individual residual components can be different. The use of a plurality of residual components that implement different dilation rates can determine interactions between amino acids located at positions of the protein sequences that are separated by at least a threshold number of intervening positions. In various examples, the use of a plurality of residual components that implement different dilation rates can determine interactions between amino acids that can be located many positions from one another in a one-dimensional sequence, yet are located proximate to one another in the protein molecule due to secondary structures and/or tertiary structures present in the protein molecule, such as a turn or fold.

In one or more illustrative examples, the systems, techniques, and methods described here can be implemented to determine sequences of antibodies. In various examples, a base sequence of an antibody can be used to generate one or more variant sequences of the antibody. The one or more variant sequences can have characteristics that are improved with respect to the base sequence. For example, the systems, techniques, and methods described herein can determine an amount of loss with respect to amino acids located in a number of positions of an antibody and generate variant sequences by replacing the amino acids associated with the highest amounts of loss. As a result, the variant sequences of the antibody can minimize an amount of loss related to the variant sequences with respect to the base sequence. In one or more examples, the loss of variant sequences can be minimized to improve biophysical properties of the antibodies having the variant sequences.

The techniques, systems, and methods described herein can generate amino acid sequences of variant proteins that have improved characteristics with respect to base sequences. For example, the techniques, systems, and methods described herein can modify an amino acid sequence of a protein having high affinity with respect to an additional protein, but also having low stability measurements and low expression levels to generate a variant amino acid sequence having high affinity with respect to the additional protein, higher stability measurements than a protein having the base sequence, and higher expression levels than a protein having the base sequence. In various examples, the techniques, systems, and methods describe herein can identify additional positions of an amino acid sequence that are candidates for substitution that are not identified using existing techniques.

As used herein, structural features of proteins can refer to features of one or more amino acids or features of one or more groups of amino acids included in a protein molecule. Examples of structural features can include at least one of hydrophobic regions that include one or more amino acids, negatively charged regions that include one or more amino acids, positively charged regions that include one or more amino acids, basic regions that include one or more amino acids, acidic regions that include one or more amino acids, regions that include one or more aromatic amino acids, neutral regions that include one or more amino acids, a measure of diversity of neighboring residues, a measure of residues interacting in ionic bonds, or regions of amino acids participating in at least one of an a-helix, a R-turn, a P-sheet, or an Q-loop. In addition, as used herein biophysical properties of proteins can refer to characteristics that can be measured using a number of analytical techniques with respect to a protein molecule. Examples of biophysical properties of proteins can include at least one of melting temperature, unfolding temperature, measures of aggregation, measures of stability, measures of molecular weight, measures of interactions between regions as determine by self-interaction nanoparticle spectroscopy (SINS), measures of viscosity, pH values, or measures of solubility.

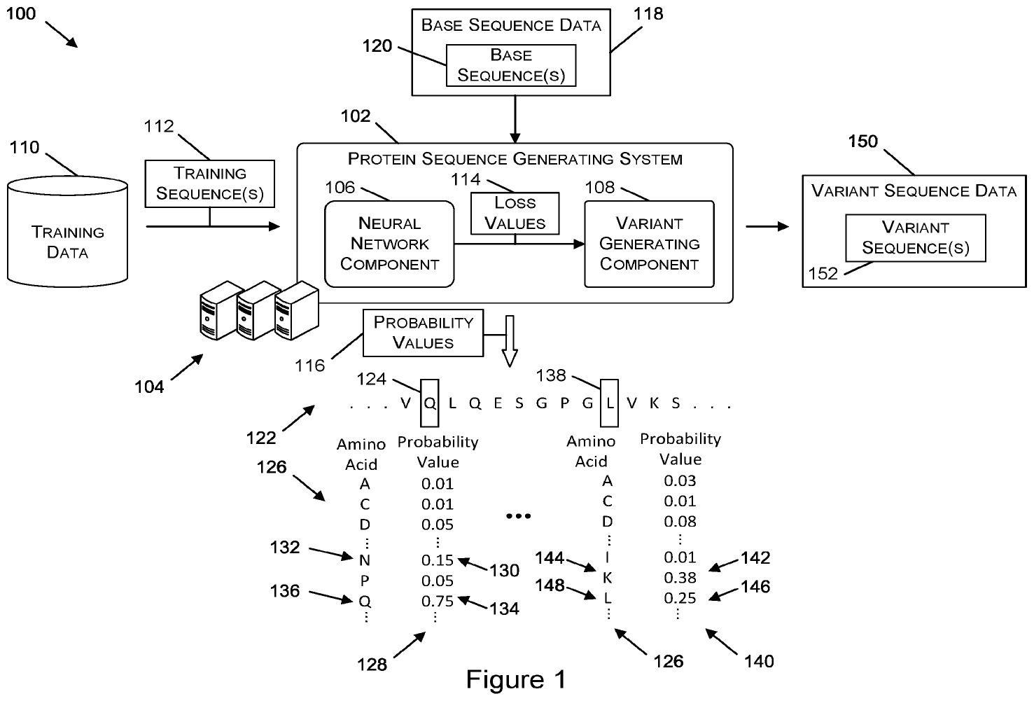

FIG. 1 is a diagram illustrating an example framework 100 to generate protein sequences based on loss values for a base sequence, in accordance with one or more implementations. The framework 100 can include a protein generating system 102. The protein sequence generating system 102 can be implemented by one or more computing devices 104. The one or more computing devices 104 can include one or more server computing devices, one or more desktop computing devices, one or more laptop computing devices, one or more tablet computing devices, one or more mobile computing devices, or combinations thereof. In certain implementations, at least a portion of the one or more computing devices 104 can be implemented in a distributed computing environment. For example, at least a portion of the one or more computing devices 104 can be implemented in a cloud computing architecture.

The protein generating system 102 can implement one or more machine learning techniques to generate amino acid sequences of variant proteins based on amino acid sequences of base proteins. For example, the protein generating system 102 can include a neural network component 106. The neural network component 106 can implement one or more convolutional neural networks to generate data that is used to produce amino acid sequences of variant proteins based on amino acid sequences of base proteins. In one or more examples, the neural network component 106 can implement one or more one-dimensional convolutional neural networks to generate probabilities of amino acids being located at given positions of a base amino acid sequence. The probabilities can then be used to generate amino acid sequences of variant proteins based on amino acid sequences of base proteins. The implementation of convolutional neural networks by the neural network component 106 can result in more efficient use of processing resources and more accurate classification probabilities than other machine learning techniques. Additionally, the one or more convolutional neural networks of the neural network component 106 can include a number of residual blocks. The output from individual residual blocks can be fed as input into a subsequent residual block. In various examples, the use of residual blocks as part of convolutional neural networks of the neural network component 106 can help to improve the efficiency of the neural network component 106 by reducing the amount of time to train the neural network component 106 in relation to convolutional neural networks that do not include residual blocks.

The protein generating system 102 can also include a variant generating component 108. The variant generating component 108 can obtain information from the neural network component 106 and generate amino acid sequences of variant proteins based on amino acid sequences of base proteins. In various examples, the variant generating component 108 can replace one or more amino acids at one or more respective positions of an amino acid sequence of a base protein to generate an amino acid sequence of a variant protein. In one or more examples, the variant generating component 108 can generate amino acid sequences of variant proteins that have different values of biophysical properties than base proteins. In addition, the variant generating component 108 can generate amino acid sequences of variant proteins that have different structure features than base proteins. Further, the variant generating component 108 can generate amino acid sequences of variant proteins that have been humanized with respect to base proteins. To illustrate, the variant generating component 108 can replace amino acids of base proteins that are produced by a mammal other than a human with additional amino acids at a number of positions such that the amino acid sequences of the variant proteins have a greater amount of homology with respect to corresponding human proteins than proteins produced by the mammal other than a human. In still other examples, the variant generating component 108 can generate amino acid sequences of variant proteins that have a greater amount of homology with respect to a protein produced in relation to a respective germline than an amino acid sequence of a base protein.

The neural network component 106 can undergo a training process with respect to training data 110. The training data 110 can be stored by a data repository that is accessible to the protein sequence generating system 102. The training data 110 can include amino acid sequences of a number of proteins. In one or more examples, the training data 110 can include amino acid sequences of proteins stored in a publicly available data repository. In one or more illustrative examples, the training data 110 can include amino acid sequences of antibodies stored by the Observed Antibody Space (OAS) database. The training data 110 can also be generated using one or more additional machine learning techniques. In one or more illustrative examples, the training data 110 can include amino acid sequences generated by a generative adversarial network (GAN).

The training data 110 can include amino acid sequences of proteins having one or more characteristics. The one or more characteristics can include at least one of biophysical properties or structural features. In various examples, the one or more characteristics can be related to stability of proteins. In one or more additional examples, the one or more characteristics can be related to yield of proteins as a product of a biomanufacturing process. The training data can be generated by filtering amino acid sequences of proteins based on one or more criteria to determine a subset of proteins having one or more characteristics that correspond to the one or more criteria. Additionally, the training data 110 can be generated by one or more generative adversarial networks that has been trained using amino acid sequences having one or more specified characteristics. In one or more examples, the one or more generative adversarial networks can be trained using one or more transfer learning techniques that utilize amino acid sequences of proteins having a set of one or more specified characteristics.

In one or more examples, the training data 110 can include amino acid sequences of proteins having a specified range of solubility values in water. In one or more additional illustrative examples, the training data 110 can include amino acid sequences of proteins having at least one region that includes at least a threshold number of hydrophobic amino acids. In one or more further illustrative examples, the training data 110 can include amino acid sequences of proteins derived from one or more germline genes. The training data 110 can also include amino acid sequences of proteins produced by a specified organism. For example, the training data 110 can include amino acid sequences of proteins produced by one or more mammals. In one or more illustrative examples, the training data 110 can include amino acid sequences of proteins produced by humans. In various examples, the training data 110 can include amino acid sequences of antibodies. Additionally, the training data 110 can include amino acid sequences of antibody fragments. The antibody fragments can include at least a portion of antibody light chains, at least a portion of antibody heavy chains, at least a portion of antigen binding regions, at least a portion of complementarity determining regions (CDRs), or one or more combinations thereof. Further, the training data 110 can include amino acid sequences of fibronectin type III (FNIII) proteins, avimers, VHH domains, kinases, zinc fingers, T-cell receptors, one or more combinations thereof, and the like.

The protein sequence generating system 102 can obtain training sequences 112. The training sequences 112 can be a subset of the amino acid sequences included in the training data 110. The training sequences 112 can be used to train the neural network component 106. In one or more examples, thousands, tens of thousands, up to millions of amino acid sequences can be included in the training sequences 112. The training sequences 112 can train the neural network component 106 to determine loss values 114 that are provided to the variant generating component 108. The loss values 114 can correspond to individual amino acids included in an amino acid sequence. For example, the loss values 114 can correspond to probability values 116 of amino acids being located at respective positions of an amino acid sequence based on the training sequences 112. In one or more illustrative examples, the neural network component 106 can include one or more convolutional neural networks. In these scenarios, the training sequences 112 can be used to train one or more kernels of the convolutional neural networks. In various examples, the neural network component 106 can implement one or more kernels that correspond to a group of amino acids, such as a group of three amino acids or a group of five amino acids. The neural network component 106 can also implement an individual kernel to determine a probability of an individual amino acid being present at one or more positions of an amino acid sequence.

After training of the neural network component 106 using the training sequences 112, the protein sequence generating system 102 can obtain base sequence data 118. The sequence data 118 can include one or more base sequences 120. The one or more base sequences 120 can correspond to amino acid sequences of proteins that are to be evaluated by the protein sequence generating system 102. For example, the neural network component 106 can determine probability values 116 indicating probabilities for individual amino acids being located at one or more respective positions of the one or more base sequences 120. The probabilities can be used by the neural network component 106 to determine the loss values 114. To illustrate, lower probability values 116 for an amino acid located at a position of a base sequence 120 can result in an increased loss value 114 for the position. Additionally, higher probability values of amino acids being located at given positions of a base sequence 120 can result in lower loss values 114 at the given positions.

In various examples, the loss values 114 can be generated by at least one of computational logic or computer-readable instructions that are included in the neural network component 106. That is, in addition to the neural network component 106 including at least one of computational logic or computer-readable instructions to execute implementations of neural networks, the neural network component 106 can include at least one of additional computational logic or additional computer-readable instructions to determine the loss values 114 from probability values generated by the neural network computational logic and/or neural network computer-readable instructions. In one or more further examples, the at least one of computational logic or computer-readable instructions that generate the loss values 114 from the probability values determined by the neural network computational logic and/or the neural network computer-readable instructions can be separate from the neural network component 106. To illustrate, in at least some examples, at least a portion of the computational logic and/or computer-readable instructions that generate the loss values 114 can be included in the variant generating component 108.

In one or more examples, the protein sequence generating system 102 can obtain base sequence data 118 that includes an illustrative base sequence 122. The neural network component 106 can analyze individual positions of the illustrative base sequence 122 in relation to the training sequences 112. In various examples, the neural network component 106 can include one or more convolutional neural networks and one or more kernels of the neural network component 106 can be used to determine the probabilities of amino acids being present at respective positions of the illustrative base sequence 122. In one or more illustrative examples, the neural network component 106 can analyze an amino acid located at a first position 124 of the illustrative base sequence 122. To illustrate, the neural network component 106 can analyze the amino acid located at the first position 124 with respect to a group of amino acids 126. The group of amino acids 126 can correspond to at least a portion of proteinogenic a-amino acids found in eukaryotes. For example, the group of amino acids 126 can include at least one of arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, or tryptophan.

In one or more examples, the neural network component 106 can determine a first group of probability values 128 of the group of amino acids 126 being present at the first location 124. The first group of probability values 128 can include, among others, a first probability value 130 that corresponds to a first amino acid 132 of the group of amino acids 126 and a second probability value 134 that corresponds to a second amino acid 136 of the group of amino acids 126. The neural network component 106 can determine a loss value 114 with respect to the first position 124 based on the first group of probability values 128. In the illustrative example of FIG. 1, the second amino acid 136 is the same as the amino acid located at the first position 124 and the second probability value 134 has a highest magnitude of the first group of probability values 128. In this illustrative scenario, the neural network component 106 can determine a loss value 114 for the first position 124 based on the magnitude of the second probability value 134 and/or based on the magnitude of the second probability value 134 in relation to the additional probability values of the first group of probability values 128.

Additionally, the neural network component 106 can analyze an amino acid located at a second position 138 of the illustrative base sequence 122. For example, the neural network component 106 can analyze the amino acid located at the second position 138 with respect to the group of amino acids 126. In one or more examples, the neural network component 106 can determine a second group of probability values 140 of the group of amino acids 126 being present at the second location 138. The second group of probability values 140 can include, among others, a third probability value 142 that corresponds to a third amino acid 144 of the group of amino acids 126 and a fourth probability value 146 that corresponds to a fourth amino acid 148 of the group of amino acids 126. The neural network component 106 can determine a loss value 114 with respect to the second position 138 based on the second group of probability values 140. In the illustrative example of FIG. 1, the fourth amino acid 148 is the same as the amino acid located at the second position 138. The third probability value 142 has a highest magnitude of the second group of probability values 140 and the fourth probability value 146 for the fourth amino acid 148, which corresponds to the second location 138 is lower than the third probability value 142. In this illustrative scenario, the neural network component 106 can determine a loss value 114 for the second position 138 based on the magnitude of the fourth probability value 146 and/or based on the magnitude of the fourth probability value 146 in relation to the additional probability values of the second group of probability values 140. The loss value 114 for the second position 138 can be relatively higher than the loss value 114 for the first position 124 because the magnitude of the fourth probability value 146 is lower than the magnitude of the second probability value 134. Further, the loss value 114 for the second position 138 can be relatively higher than the loss value 114 for the first position 124 because the fourth probability value 146 is not a highest probability value included in the second group of probability values 140.

The loss values 114 determined by the neural network component 106 can be provided to the variant generating component 108. The variant generating component 108 can generate variant sequence data 150 that includes one or more variant sequences 152. The one or more variant sequences 152 can include amino acid sequences of variant proteins that are generated based on the one or more base sequences 120 and the loss values 114. In one or more examples, the one or more variant sequences 152 can correspond to proteins that have values of one or more biophysical properties that are different from additional proteins that correspond to the one or more base sequences 120. Additionally, the one or more variant sequences 152 can correspond to proteins that have one or more structural features that are different from additional proteins that correspond to the one or more base sequences 120. Further, the one or more variant sequences 152 can correspond to proteins produced by humans and the one or more base sequences can correspond to proteins produced by another organism, such as a non-human mammal. In this way, the one or more variant sequences 152 can be humanized with respect to the one or more base sequences 120. The variant generating component 108 can also generate one or more variant sequences 152 that correspond to a germline based on one or more base sequences 120 that do not correspond to the germline. For example, the one or more base sequences 120 can correspond to proteins generated in accordance with a first germline and the one or more variant sequences 152 can correspond to additional proteins generated in accordance with a second germline that is different from the first germline.

In one or more examples, one or more characteristics of proteins that correspond to the one or more variant sequences 152 can be based on one or more characteristics of proteins corresponding to the training sequences 112. For example, in situations where the training sequences 112 correspond to proteins having specified values of one or more biophysical properties, at least a portion of the one or more variant sequences 152 produced by the variant generating component 108 can correspond to additional proteins having the specified values of the one or more biophysical properties. In one or more illustrative examples, the specified values of the one or more biophysical properties can correspond to a range of values, values above a threshold minimum value, or values below a threshold maximum value. In one or more additional examples, the training sequences 112 can correspond to proteins having one or more structural features and at least a portion of the one or more variant sequences 152 produced by the variant generating component 108 can correspond to additional proteins having the one or more structural features. Further, in scenarios where the training sequences 112 correspond to proteins produced based on a human genome, at least a portion of the one or more variant sequences 152 can correspond to additional proteins having characteristics of the proteins produced based on a human genome. For example, at least a portion of the one or more variant sequences 152 generated by the variant generating component 108 can have at least a threshold amount of identity with respect to amino acid sequences of proteins produced in accordance with a human genome. Additionally, the training sequences 112 can correspond to proteins generated based on one or more germline genes and at least a portion of the one or more variant sequences can correspond to additional proteins having characteristics of the proteins produced based on the one or more germline genomic regions. To illustrate, at least a portion of the one or more variant sequences 152 produced by the variant generating component 108 can have at least a threshold amount of identity with respect to amino acid sequences of proteins produced based on the one or more germline genomic regions.

In various examples, the variant generating component 108 can produce the one or more variant sequences 152 by replacing an amino acid at one or more positions of the one or more base sequences 120. The variant generating component 108 can determine that one or more amino acids of a base sequence 120 are to be replaced to generate a corresponding variant sequence 152 based on one or more loss values 114 for the base sequence 120. For example, the variant generating component 108 can determine an overall loss value for the base sequence 120 that is comprised of individual loss values 114 for respective positions of the base sequence 120. In situations where the variant generating component 108 determines that the overall loss value for the base sequence 120 is greater than a threshold loss value, the variant generating component 108 can produce a variant sequence 152 by modifying amino acids located at one or more positions of the base sequence 120. The threshold loss value can be different for different characteristics. For example, the threshold loss value for proteins having specified measurements for solubility in water can be different than a threshold loss value for proteins having a negatively charged patch of a specified size.

The variant generating component 108 can determine positions of a base sequence 120 that can be modified in order to produce a variant sequence 152 that corresponds to the base sequence 120. In one or more examples, the variant generating component 108 can analyze loss values 114 of individual positions of a base sequence 120 to determine amino acids to modify in the base sequence 120 to produce a corresponding variant sequence 152. In various examples, the variant generating component 108 can identify positions of a base sequence 120 that are candidates for being modified by determining that the positions have a loss value 114 that is greater than a threshold loss value. In one or more illustrative examples, the threshold loss value used to determine whether an amino acid located at a given position is to be replaced can be determined based on probability values 116 of amino acids being located at individual positions of a base sequence 120. To illustrate, a threshold loss value can correspond to a probability value of a current amino acid located at a position of a base sequence 120 being less than a probability value of at least one candidate amino acid for the position. The variant generating component 108 can determine a replacement amino acid from among candidate amino acids for a given position of a base sequence 120 by determining a candidate amino acid having a highest probability value for the given position. In one or more additional examples, the variant generating component 108 can determine a replacement amino acid from among candidate amino acids for a given position of a base sequence 120 by determining a candidate amino acid that maximizes a characteristic or a group of characteristics of a protein having the modified sequence.

In one or more examples, the protein sequence generating system 102 can determine variant sequences 152 through an iterative process. In various examples, an iterative process can be performed because a modification to an amino acid at one position of a base sequence 120 can affect the loss values 114 for additional positions of the base sequence 120. Thus, the protein sequence generating system 102 can determine one or more proposed modifications to a base sequence 120 that minimize at least one of individual loss values or an aggregate loss value for the modified base sequence. For example, in response to determining that an amino acid located at a position of a base sequence 120 is to be changed, the protein sequence generating system 102 can generate a variant sequence 152 with the proposed modification and use the variant sequence 152 as a new base sequence. In this way, the proposed modification can be treated as temporary within the protein sequence generating component 102. The protein sequence generating component 102 can then analyze the loss values 114 for the new base sequence. In situations where the loss values 114 for the new base sequence indicate that further changes to one or more positions are to be made, the protein sequence generating system 102 can cause the new base sequence to revert to the original base sequence and to be analyzed further for one or more additional modifications to be made to the original base sequence. In scenarios where a modification to the original base sequence does not result in individual loss values and/or an aggregate loss value for the original base sequence that necessitate further changes, a status of the proposed modification can be changed from temporary to fixed. The protein sequence generating component 102 can perform a number of iterations with respect to proposed modifications until the loss values 114 for a variant sequence 152 have satisfied one or more criteria. To illustrate, the protein sequence generating component 102 can evaluate one or more proposed modifications iteratively until an amount of change in at least one of individual loss values 114 or aggregate loss levels satisfies one or more criteria. In one or more illustrative examples, the one or more criteria for determining when to cease an evaluation of one or more proposed modifications to a base sequence 120 can include an amount of change of at least one of individual loss values 114 or aggregate loss values being less than a threshold amount of change and/or the rate of change of the individual loss values 114 or aggregate loss values being less than a threshold rate of change.

In one or more illustrative examples, the variant generating component 108 can analyze the illustrative base sequence 122 to identify one or more amino acids at individuals positions of the illustrative base sequence 122 that can be replaced to generate a corresponding variant sequence. For example, the variant generating component 108 can analyze a loss value 114 of the illustrative base sequence 120 for the first position 124. The loss value 114 for the first position 124 can be determined by analyzing a probability for a current amino acid located at the first position 124 with respect to a maximum probability of an amino acid included in the group of amino acids 126 for the first position 124. In the illustrative example of FIG. 1, the current amino acid at the first position 124 is a glutamine (Q) molecule and has a probability value of 0.75 that is the maximum probability value for amino acids with respect to the first position 124. As a result, the loss value 114 for the first position 124 is minimized and the variant generating component 108 can determine that the glutamine amino acid located at the first position 124 is not to be replaced.

Additionally, the variant generating component 108 can analyze a loss value 114 of the illustrative base sequence 122 for the second position 138. The loss value 114 for the second position 138 can be determined by analyzing a probability for a current amino acid located at the second position 138 with respect to a maximum probability of an amino acid included in the group of amino acids 126 for the second position 138. In the illustrative example of FIG. 1, the current amino acid at the second position 138 is a leucine (L) molecule and has a probability value of 0.25. The probability value for the leucine molecule at the second position 138 is less than the probability value of 0.38 for a lysine (K) molecule at the second position 138. As a result, the variant generating component 108 can determine that the leucine molecule currently located at the second position 138 of the illustrative base sequence 122 is to be replaced by a lysine molecule. In these scenarios, the variant generating component 108 can generate a variant sequence based on the illustrative base sequence 122 where a lysine molecule is present at the second position 138 rather than a leucine molecule.

In one or more examples, the protein sequence generating system 102 can include a number of neural network components 106 and/or a number of variant generating components 108. In one or more illustrative examples, the protein sequence generating system 102 can include multiple neural network components 106 with each neural network component 106 being trained to evaluate amino acid sequences, such as the base sequences 120, with respect to one or more biophysical properties of proteins and/or one or more structural features of proteins. For example, the protein sequence generating system 102 can include a first neural network component that generates first loss values with respect to a size of hydrophobic patches of proteins that correspond to one or more base sequences 120, a second neural network component that generates second loss values with respect to size of negatively charged patches of proteins that correspond to one or more base sequences 120, and a third neural network component that generates third loss values with respect to a melting temperature of proteins that correspond to one or more base sequences 120. In these scenarios, the protein generating system 102 can implement a model that evaluates the first loss values, the second loss values, and the third loss values to generate an aggregate quantitative measure. The aggregate quantitative measure can correspond to a suitability of a given protein that corresponds to an individual base sequence 120 for a given purpose or function, such as binding to a target molecule or stability in a given environment

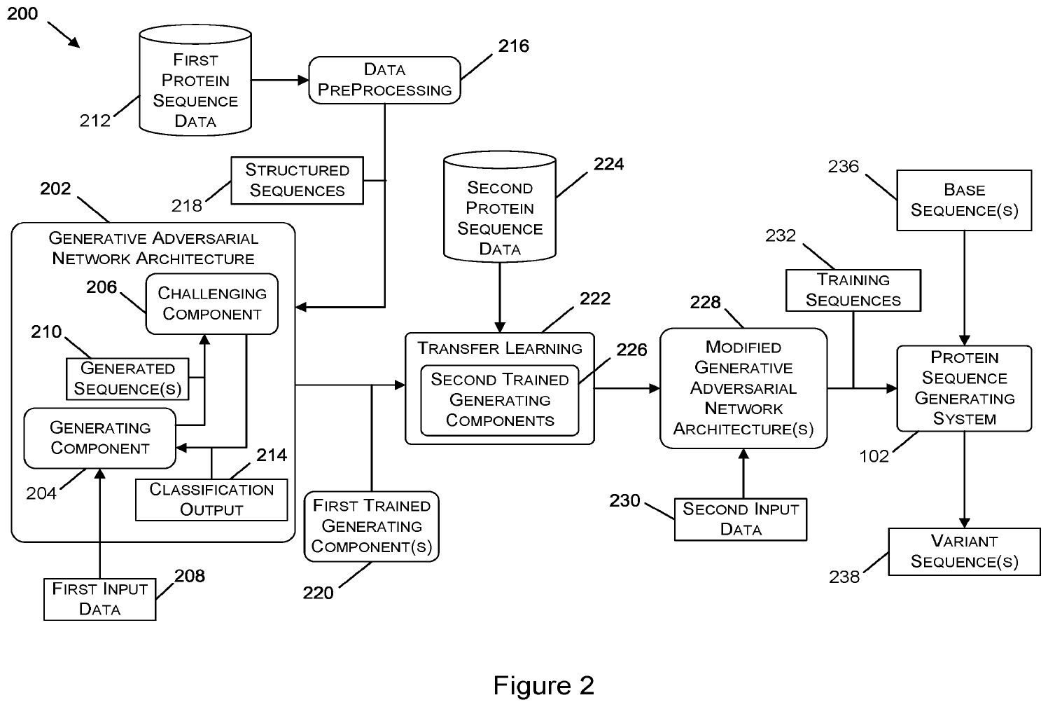

In one or more illustrative examples, the individual neural network components can be trained according to one or more transfer learning techniques using amino acid sequences of proteins that correspond to the one or more biophysical properties and/or the one or more structural features that an individual neural network component is evaluating. Continuing with the example from above, the first neural network component can be trained using first amino acid sequences that correspond to proteins having one or more sizes of hydrophobic patches. Additionally, the second neural network component can be trained using second amino acid sequences that correspond to additional proteins having one or more sizes of negatively charged patches. Further, the third neural network component can be trained using third amino acid sequences that correspond to further proteins having one or more melting temperatures or one or more ranges of melting temperatures. In at least some examples, at least one of the first neural network component, the second neural network component, or the third neural network component can be trained according to at least a portion of the operations described with respect to FIG. 2. Examples of biophysical properties, structural features, or protein characteristics that can be evaluated by individual neural network components can include large or small surface patch sizes, ionic bond formation, or modified hydrogen bonding networks, or generalized properties, such as changed thermal characteristics, chemical characteristics, colloidal stability, immunogenicity, pK (through surrogate assays), or target binding energies.

In various examples, the loss values generated by the different neural network components can be weighted by the model used to determine the aggregate quantitative measure. The weighting of the loss values generated by individual neural network components can be based on a characteristic of proteins that is being evaluated by the protein sequence generating system 102. Additionally, the loss values for a set of structure features and/or biophysical properties that are used to generate the aggregate quantitative measure can also be based on a characteristic of proteins that is being evaluated by the protein sequence generating system 102. For example, a first set of structural features and/or biophysical properties can be used to evaluate amino acid sequences with respect to solubility in a given environment and a second set of structural features and/or biophysical properties can be used to evaluate amino acid sequences with respect to binding to a given target molecule. In one or more examples, the weights for the loss values generated by individual neural network components can be determined using one or more machine learning techniques and training data that includes amino acid sequences of proteins that have the desired characteristics. In one or more illustrative examples, a reinforcement learning machine learning architecture can be implemented to determine one or more models for evaluating outputs obtained from multiple neural network components in relation to a given desired characteristic of proteins.

In one or more additional examples, the loss values generated by multiple neural network components can be provided to the variant generating component 108. The variant generating component 108 can then optimize changes to the base sequences 120 to generate variant sequences 152 that are optimized for one or more biophysical properties and/or one or more structural features. To illustrate, the variant generating component 108 can determine one or more first modifications to a base sequence 120 to modify a first property of the base sequence 120 based on loss scores obtained from a first neural network component and one or more second modifications to the base sequence 120 to modify a second property of the base sequence 120 based on additional loss scores obtained from a second neural network component. The variant generating component 108 can then determine whether or not one or more of the first modifications adversely affect a first biophysical property or first structural feature of the second neural network component or whether or not one or more of the second modifications adversely affect a second biophysical property or second structural feature of the first neural network component. Based on changes to the values for the first and/or second biophysical properties or structural features, the variant generating component 108 can determine whether or not to make a given modification to an amino acid of a base sequence 120 when generating a corresponding variant sequence 152.

FIG. 2 is a diagram illustrating an example framework 200 to train a generative adversarial network using transfer learning techniques to generate training data for a protein sequence generating system 102 that includes a neural network component, in accordance with one or more implementations. The framework 200 can include a generative adversarial network architecture 202. The generative adversarial network architecture 202 can include a generating component 204 and a challenging component 206. The generating component 204 can implement one or more models to generate amino acid sequences based on input provided to the generating component 204. In various implementations, the one or more models implemented by the generating component 204 can include one or more functions and one or more weights. The challenging component 206 can generate output indicating whether the amino acid sequences produced by the generating component 204 correspond to various characteristics. The output produced by the challenging component 206 can be provided to the generating component 204 and the one or more models implemented by the generating component 204 can be modified based on the feedback provided by the challenging component 206. In various implementations, the challenging component 206 can analyze the amino acid sequences generated by the generating component 204 with amino acid sequences of proteins included in training data and generate an output indicating an amount of correspondence between the amino acid sequences produced by the generating component 204 and the amino acid sequences of proteins provided to the challenging component 206 as training data. In one or more illustrative examples, the analysis performed by the challenging component 206 with respect to the amino acid sequences produced by the generating component 204 can include a comparison between the amino acid sequences included in the training data and the amino acid sequences produced by the generating component 204.

In various implementations, the generative adversarial network architecture 202 can implement one or more neural network technologies. For example, the generative adversarial network architecture 202 can implement one or more recurrent neural networks. Additionally, the generative adversarial network architecture 202 can implement one or more convolutional neural networks. In one or more implementations, the generative adversarial network architecture 202 can implement a combination of recurrent neural networks and convolutional neural networks. In one or more additional examples, the generating component 204 can include a generator and the challenging component 206 can include a discriminator. In one or more further implementations, the generative adversarial network architecture 202 can include a Wasserstein generative adversarial network (wGAN). In these scenarios, the generating component 204 can include a generator and the classifying component 206 can include a critic.

In the illustrative example of FIG. 2, first input data 208 can be provided to the generating component 204 and the generating component 204 can produce one or more generated sequences 210 from the first input data 208 using one or more models. In one or more implementations, the first input data 208 can include a vector comprised of noise data that is generated by a random number generator or a pseudo-random number generator. The generated sequence(s) 210 can be compared by the challenging component 206 against sequences of proteins included in first protein sequence data 212 that have been structured according to one or more schemas. The first protein sequence data 212 can include sequences of proteins obtained from one or more data sources that store amino acid sequences of proteins. The first protein sequence data 212 can be training data for the generative adversarial network architecture 202.

Based on similarities and/or differences between the generated sequence(s) 210 and the sequences obtained from the first protein sequence data 212, the challenging component 206 can generate a classification output 214 that indicates an amount of similarity and/or an amount of difference between the generated sequence 210 and sequences included in the first protein sequence data 212. In one or more examples, the challenging component 206 can label the generated sequence(s) 210 as zero and the sequences obtained from the first protein sequence data 212 can be labeled as one. In these situations, the classification output 214 can correspond to a number from 0 and 1. In additional examples, the challenging component 206 can implement a distance function that produces an output that indicates an amount of distance between the generated sequence(s) 210 and the proteins included in the first protein sequence data 212. In these scenarios, the challenging component 206 can label the generated sequence(s) 210 as −1 and the encoded amino acid sequences obtained from the protein sequence data 212 as 1. In implementations where the challenging component 206 implements a distance function, the classification output 214 can be a number from −∞ to ∞. In various examples, the amino acid sequences obtained from the first protein sequence data 212 can be referred to as ground truth data.

The protein sequences included in the first protein sequence data 212 can be subject to data preprocessing 216 before being provided to the challenging component 206. In one or more implementations, the first protein sequence data 212 can be arranged according to a classification system before being provided to the challenging component 206. The data preprocessing 216 can include pairing amino acids included in the proteins of the first protein sequence data 212 with numerical values that can represent structure-based positions within the proteins. The numerical values can include a sequence of numbers having a starting point and an ending point. In an illustrative example, a T can be paired with the number 43 indicating that a Threonine molecule is located at a structure-based position 43 of a specified protein domain type. In one or more illustrative examples, structure-based numbering can be applied to any general protein type, such as fibronectin type III (FNIII) proteins, avimers, antibodies, VHH domains, kinases, zinc fingers, and the like.

In one or more implementations, the classification system implemented by the data preprocessing 216 can designate a particular number of positions for certain regions of proteins. For example, the classification system can designate that portions of proteins having particular functions and/or characteristics can have a specified number of positions. In various situations, not all of the positions included in the classification system may be associated with an amino acid because the number of amino acids in a specified region of a protein can vary between proteins. To illustrate, the number of amino acids in a region of a protein can vary for different types of proteins. In one or more examples, positions of the classification system that are not associated with a particular amino acid can indicate various structural features of a protein, such as a turn or a loop. In an illustrative example, a classification system for antibodies can indicate that heavy chain regions, light chain regions, and hinge regions have a specified number of positions assigned to them and the amino acids of the antibodies can be assigned to the positions according to the classification system.

The data used to train the generative adversarial network architecture 202 can impact the amino acid sequences produced by the generating component 204. For example, in situations where antibodies are included in the first protein sequence data 212 provided to the challenging component 206, the amino acid sequences generated by the generating component 204 can correspond to antibody amino acid sequences. In another example, in scenarios where T-cell receptors are included in the first protein sequence data 212 provided to the challenging component 206 the amino acid sequences generated by the generating component 204 can correspond to T-cell receptor amino acid sequences. In one or more additional examples, in situations where kinases are included in the first protein sequence data 212 provided to the challenging component 206, the amino acid sequences generated by the generating component 204 can correspond to amino acid sequences of kinases. In implementations where amino acid sequences of a variety of different types of proteins are included in the first protein sequence data 212 provided to the classifying component 206, the generating component 204 can generate amino acid sequences having characteristics of proteins generally and may not correspond to a particular type of protein.

The output produced by the data preprocessing 216 can include structured sequences 218. The structured sequences 218 can include a matrix indicating amino acids associated with various positions of a protein. In one or more examples, the structured sequences 218 can include a matrix having columns corresponding to different amino acids and rows that correspond to structure-based positions of proteins. For each element in the matrix, a 0 can be used to indicate the absence of an amino acid at the corresponding position and a 1 can be used to indicate the presence of an amino acid at the corresponding position. In situations where a position represents a gap in an amino acid sequence, the row associated with the position can comprise zeroes for each column. The generated sequence(s) 210 can also be represented using a vector according to a same or similar number scheme as used for the structured sequences 218. In one or more illustrative examples, the structured sequences 218 and the generated sequence(s) 210 can be encoded using a method that may be referred to as a one-hot encoding method.

After the generative adversarial network architecture 202 has undergone a training process, one or more first trained generating components 220 can be generated that can produce amino acid sequences of proteins. In one or more examples, the training process for the generative adversarial network architecture 202 can be complete after the function(s) implemented by the generating component 204 and the function(s) implemented by the challenging component 206 converge. The convergence of a function can be based on the movement of values of model parameters toward specified values as protein sequences are generated by the generating component 204 and feedback is obtained from the challenging component 206. In various implementations, the training of the generative adversarial network architecture 202 can be complete when the protein sequences generated by the generating component 204 have one or more specified characteristics. To illustrate, the amino acid sequences generated by the generating component 204 can be analyzed by a software tool that can analyze amino acid sequences to determine at least one of biophysical properties of the amino acid sequences, structural features of the amino acid sequences, or adherence to amino acid sequences corresponding to one or more protein germlines.

The one or more first trained generating components 220 can be included in a transfer learning process 222. The transfer learning process 222 can be performed with respect to the one or more first trained generating components 220 and second protein sequence data 224. The transfer learning that is performed at 222 can modify the one or more first trained generating components 220 based on the amino acid sequences included in the second protein sequence data 224. The transfer learning that is performed at 222 can produce one or more second trained generating components 226 that are modified versions of the one or more first trained generating components 220. In various examples, the transfer learning that takes place at 222 can include an additional training process of the one or more first trained generating components 220 using training data obtained from the second protein sequence data 224. By using a training dataset to produce the second trained generating components 226 that is different from the training dataset used to produce the first trained generating components 220 the second trained generating components 226 can produce amino acid sequences that can have some general characteristics that correspond to the amino acid sequences included in the first protein sequence data 212 and that also have one or more specified characteristics that correspond to features of the proteins related to the amino acid sequences included in the second protein sequence data 224. In one or more examples, the number of amino acid sequences included in at least one of the first protein sequence data 212 or the second protein sequence data 224 can be in the thousands of amino acid sequences up to tens of thousands of amino acid sequences.

In various implementations, the one or more first trained generating components 220 can be trained to produce the one or more second trained generating components 226 in a manner that is similar to the training of the generative machine learning architecture 202 that produced the one or more first trained generating components 220. In one or more examples, at least one of parameters, weights, or other model features of the one or more first trained generating components 220 can be modified to minimize at least one loss function. Additionally, the training process for the one or more first generating components 220 used to produce the one or more second trained generating components 226 can be complete after one or more functions implemented by the one or more second trained generating components 226 converge. In one or more further examples, the training process used to generate the one or more second trained generating components 226 from the one or more first trained generating components 220 can be complete based on an analysis of a software tool indicating that amino acid sequences produced using the one or more second trained generating components 226 corresponds to one or more specified criteria. The one or more specified criteria can correspond to at least one of one or more structural features of proteins having amino acid sequences produced by the one or more second trained generating components 226 or one or more biophysical properties of proteins having amino acid sequences produced by the one or more second trained generating components 226.

In one or more examples, the second protein sequence data 224 can include amino acid sequences of proteins that have features that are different from the features of the proteins related to the first protein sequence data 212. In various examples, the second protein sequence data 224 can include a subset of the amino acid sequences included in the first protein sequence data 212. In additional examples, the second protein sequence data 224 can include a greater number of a group of amino acid sequences having one or more specified characteristics in relation to the number of amino acid sequences having the one or more specified characteristics included in the first protein sequence data 212. For example, the first protein sequence data 212 can include amino acid sequences of proteins having a variety of structural features. To illustrate, the first protein sequence data 212 can include a number of amino acid sequences of proteins having various sizes of hydrophobic regions, a number of amino acid sequences of proteins various sizes of negatively charged regions, a number of amino acid sequences of proteins having various sizes of positively charged regions, a number of amino acid sequences of proteins various sizes of polar regions, one or more combinations thereof, and the like. Additionally, the second protein sequence data 224 can include a greater number and/or greater percentage of amino acid sequences of proteins that have hydrophobic regions with a specified range of sizes than the number of amino acid sequences included in the first protein sequence data 212 that have the hydrophobic regions with the specified range of sizes. In these scenarios, the second trained generating components 226 can primarily produce amino acid sequences of proteins having hydrophobic regions with the specified range of sizes. In further examples, the second protein sequence data 224 can include a greater number and/or greater percentage of amino acid sequences of proteins that have negatively charged regions with a specified range of sizes than the number of amino acid sequences included in the first protein sequence data 212 that have negatively charged regions with the specified range of sizes. In these situations, the one or more second trained generating components 226 can primarily produce amino acid sequences of proteins having negatively charged regions with the specified range of sizes.

In one or more implementations, the amino acid sequences included in the second protein sequence data 224 can include a filtered set of amino acid sequences. For example, a set of amino acid sequences can be evaluated according to one or more criteria. In various examples, at least one of one or more software tools, one or more diagnostic tools, or one or more analytical instruments can be used to identify amino acid sequences included in the set of amino acid sequences that correspond to the one or more criteria. The amino acid sequences that satisfy the one or more criteria can then be added to the second protein sequence data 224. In one or more illustrative examples, a number of amino acid sequences can be evaluated to identify proteins having at least one polar region for inclusion in the second protein sequence data 224 that can be used to modify the one or more first trained generating components 220 to produce the one or more second trained generating components 226. In one or more additional illustrative examples, a number of amino acid sequences can be evaluated to identify proteins including at least one positively charged region having a specified range of sizes for inclusion in the second protein sequence data 224 that can be used to modify the one or more first trained generating components 220 to produce the one or more second trained generating components 226.

In one or more illustrative examples, the transfer learning at 222 can modify a first distribution of proteins having one or more characteristics produced by the one or more first trained generating components 220 such that a second distribution of proteins having the one or more characteristics produced by the one or more second trained generating components 226 is different from the first distribution. For example, the one or more first trained generating components 220 can produce proteins having at least one hydrophobic region included in a first range of sizes, such as from about 1 amino acid to about 15 amino acids with an average size of 7 amino acids and a standard deviation of 1.5 amino acids. Additionally, the one or more second trained generating components 226 can produce proteins having at least one hydrophobic region includes in a second range of sizes, such as from about 9 amino acids to about 15 amino acids with an average size of 12 amino acids and a standard deviation of 0.5 amino acids. In these scenarios, the second protein sequence data 224 can include amino acid sequences of proteins having at least one hydrophobic region that corresponds to the second range of sizes. In one or more additional examples, a probability of the one or more first trained generating components 220 producing proteins having one or more characteristics can be different from a probability of the one or more second trained generating components 226 producing proteins having the one or more characteristics. To illustrate, the one or more first trained generating components 220 can have from about a 10% probability to about a 15% probability of generating amino acid sequences of proteins having at least one negatively charged region with no greater than 5 amino acids, while the one or more second trained generating components 226 can have from about a 95% probability to about a 99% probability of generating amino acid sequences of proteins having at least one negatively charged region with no greater than 5 amino acids. In these situations, the second protein sequence data 224 can include amino acid sequences of proteins having at least one negatively charged patch with a size that is no greater than 5 amino acids. The one or more second trained generating components 226 can be included in one or more modified generative machine learning architectures 228. In one or more examples, the one or more modified generative adversarial network architectures 228.

The one or more modified generative adversarial network architectures 228 can generate amino acid sequences based on second input data 230. In one or more examples, the second input data 230 can include a random or pseudo-random series of numbers that can be used by one or more generative adversarial networks included in the one or more modified generative adversarial network architectures 228 to produce amino acid sequences.

In various examples, the one or more modified generative adversarial network architectures 228 can include a plurality of generative adversarial network architectures with individual generative adversarial network architectures producing sequences of amino acids that correspond to proteins that have one or more specified structural features. For example, the one or more modified generative adversarial network architectures 228 can include a first generative adversarial network architecture that produces amino acid sequences that correspond to proteins having hydrophobic patches with a first range of sizes, a second generative adversarial network architecture that produces amino acid sequences that correspond to proteins having hydrophobic patches with a second range of sizes, and a third generative adversarial network architecture that produces amino acid sequences that correspond to proteins having hydrophobic patches with a third range of sizes. In one or more examples, there may be some overlap between the sizes of hydrophobic patches included in the first range of sizes and the second range of sizes and between sizes of hydrophobic patches included in the second range of sizes and the third range of sizes.

The amino acid sequences generated by the one or more modified generative adversarial network architectures 228 can include training sequences 232 that can be used to train a neural network component of the protein sequence generating system 102. The training sequences 232 can include amino acid sequences of proteins that have one or more characteristics that correspond to one or more characteristics of proteins related to the second protein sequence data 224, such as values of one or more biophysical properties and/or the presence or absence of one or more structural features. In various examples, the training sequences 232 can be used to train one or more kernels of one or more convolutional neural networks of the protein sequence generating system 102. After the neural network component of the protein sequence generating system 102 has been trained using the training sequences 232, the protein sequence generating system 102 can obtain one or more base sequences 236. The protein sequence generating system 102 can produce one or more variant sequences 238 that correspond to the one or more base sequences 236. In one or more examples, the protein sequence generating system 102 can modify the amino acids present at one or more locations of individual base sequences 236 to generate individual variant sequences 238. In various examples, the protein sequence generating system 102 can generate multiple variant sequences 238 from a single base sequence 236. To illustrate, the protein sequence generating system 102 can generate a first variant sequence according to an individual base sequence by making a first set of modifications to a group of first positions of the individual base sequence and a second variant sequence according to the individual base sequence by making a second set of modifications to a group of second positions of the individual base sequence.

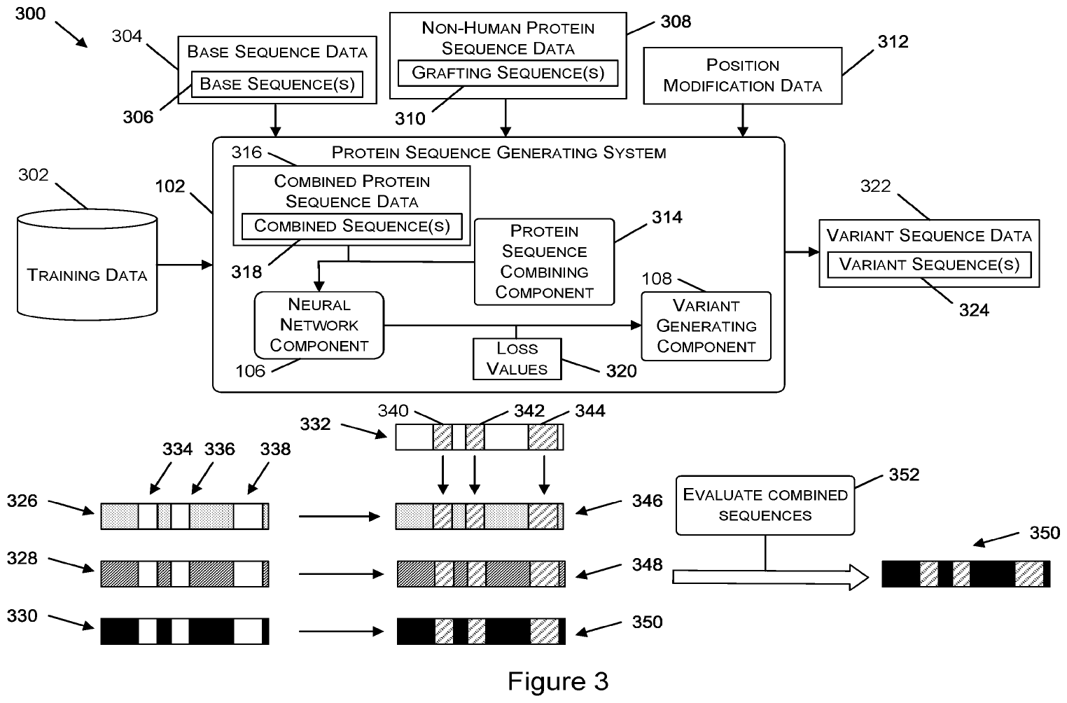

FIG. 3 is a diagram illustrating an example framework 300 to generate humanized protein sequences using human template protein sequences and supplemental non-human protein sequences, in accordance with one or more implementations. The framework 300 includes the protein sequence generating system 102. The protein sequence generating system 102 can include the neural network component 106. The neural network component 106 can be trained using training data 302. The training data 302 can include amino acid sequences of proteins. In one or more examples, the neural network component 106 can be trained using the training data 302 to identify amino acid sequences that correspond to the amino acid sequences included in the training data 302. The neural network component 106 can also be trained using the training data 302 to generate amino acid sequences that correspond to the amino acid sequences included in the training data 302. Further, the neural network component 106 can be trained using the training data 302 to generate amino acid sequences of proteins having one or more characteristics of proteins and/or protein fragments corresponding to the amino acid sequences included in the training date 302. In one or more illustrative examples, the neural network component 106 can include one or more convolutional neural networks. In these scenarios, the training data 302 can be used to train one or more kernels of the one or more convolutional neural networks.

The amino acid sequences included in the training data 302 can correspond to at least one of proteins or protein fragments generated by humans. In various examples, the amino acid sequences included in the training data 302 can have characteristics, such as biophysical properties and/or structural features, of at least one of proteins or protein fragments generated by humans. Additionally, the training data 302 can include amino acid sequences of at least one of proteins or protein fragments produced in accordance with one or more human germline genomic regions. In one or more illustrative examples, the training data 302 can include amino acid sequences of at least one of antibodies or antibody sequences generated by humans. For example, the training data 302 can include amino acid sequences of at least one of antibodies or antibody sequences generated in accordance with one or more human germline genomic regions. In one or more additional illustrative examples, the training data 302 can include amino acid sequences of fibronectin type III (FNIII) proteins, avimers, VHH domains, kinases, zinc fingers, T-cell receptors, one or more combinations thereof, and the like, generated in accordance with one or more human germline genomic regions.

The protein sequence generating system 102 can obtain base sequence data 304. The base sequence data 304 can include one or more base sequences 306. The one or more base sequences 306 can correspond to amino acid sequences of proteins that are to be analyzed by the protein sequence generating system 102. In one or more examples, the base sequence data 304 can include base sequences 306 that correspond to amino acid sequences of at least one of proteins or protein fragments generated in accordance with one or more human genomic regions. In one or more illustrative examples, the base sequence data 304 can include base sequences 306 that correspond to amino acid sequences of at least one of antibody or antibody fragments generated in accordance with one or more human genomic regions. In one or more additional illustrative examples, the base sequence data 304 can include base sequences 306 that correspond to amino acid sequences of fibronectin type III (FNIII) proteins, avimers, VHH domains, kinases, zinc fingers, T-cell receptors, one or more combinations thereof, and the like, generated in accordance with one or more human genomic regions.

Additionally, the protein sequence generating system 102 can obtain non-human protein sequence data 308. The non-human protein sequence data 308 can include amino acid sequences of at least one of proteins or protein fragments generated in accordance with non-human genomic regions. In one or more examples, the base sequence data 304 can include base sequences 306 that correspond to amino acid sequences of at least one of proteins or protein fragments generated in accordance with one or more human genomic regions. In various examples, the non-human protein sequence data 308 can include grafting sequences 310 that correspond to amino acid sequences of at least one of proteins or protein fragments that correspond to the proteins and/or protein fragments related to the base sequence data 304. For example, in scenarios where the base sequence data 304 corresponds to amino acid sequences of at least one of antibodies or antibody fragments generated in accordance with one or more human genomic regions, the grafting sequences 310 can correspond to at least one of antibodies or antibody fragments generated in accordance with one or more non-human genomic regions. In various examples, the grafting sequences 310 can be grafted into one or more locations of one or more base sequences 306. In one or more implementations, a grafting sequence 310 can be grafted into a base sequence 306 by substituting one or more amino acids of the grafting sequence 310 at one or more positions of the base sequence 306.

Further, the protein sequence generating system 102 can obtain position modification data 312. The position modification data 312 can indicate positions of at least one of the base sequences 306 or the grafting sequences 310 that are available to be modified. The position modification data 312 can be applied by one or more components of the protein sequence generating system 102. For example, the position modification data 312 can be applied by at least one of the neural network component 106 or a protein sequence combining component 314.

In one or more examples, the position modification data 312 can indicate one or more criteria related to the modification of at least one of amino acids of the one or more base sequences 306 or amino acids of the one or more grafting sequences 310. For example, the position modification data 312 can indicate one or more criteria corresponding to the modification of individual amino acids of the one or more base sequences 306 or amino acids of the one more grafting sequences 310. To illustrate, the position modification data 312 can indicate respective probabilities that amino acids at individual positions of at least one of amino acids of the one or more base sequences 306 or amino acids of the one or more grafting sequences 310 can be modified. In additional implementations, the position modification data 312 can indicate a penalty associated with the modification of amino acids at individual positions of at least one of amino acids of the one or more base sequences 306 or amino acids of the one or more grafting sequences 310. The position modification data 312 can include values or functions corresponding to the respective amino acids located at individual positions of at least one of amino acids of the one or more base sequences 306 or amino acids of the one or more grafting sequences 310.

In addition, the position modification data 312 can correspond to individual positions of at least one of the base sequences 306 or the grafting sequences 310 or to groups of positions of at least one of the base sequences 306 or the grafting sequences 310. In one or more additional examples, the position modification data 312 can indicate positions of one or more base sequences 306 that can be modified. Additionally, the position modification data 312 may indicate positions of one or more grafting sequences 310 that correspond to amino acids that are to replace amino acids of one or more base sequences 306. To illustrate, the position modification data 312 can indicate a first group of positions of heavy chain regions of a base sequence 306 having amino acids that are to be replaced by amino acids located at a second group of positions of a grafting sequence 310. In one or more illustrative examples, the second group of positions of the grafting sequence 310 can be located in a heavy chain variable region of a non-human antibody.

In one or more illustrative examples, the position modification data 312 can include criteria that reduce the probability of amino acids being modified at positions of at least one of a base sequence 306 or a grafting sequence 310 that correspond to functionality of the protein that is to be preserved in a target protein or a variant protein. For example, a penalty associated with modifying an amino acid located in a region that is attributed to functionality of a base protein can be relatively high. Additionally, the position modification data 312 can include criteria for amino acids outside of one or more regions that are attributed to functionality of a base protein that indicate increased or neutral probabilities for modification of those amino acids. To illustrate, a penalty associated with modifying an amino acid located at a position outside of a region attributed to particular functionality of a base protein can be relatively low or neutral. Further, the position modification data 312 can indicate probabilities of changing amino acids at positions of a base protein to different types of amino acids. In one or more illustrative examples, an amino acid located at a position of a template protein can have a first penalty for being changed to a first type of amino acid and a second, different penalty for being changed to a second type of amino acid. That is, in various implementations, a hydrophobic amino acid of a base protein can have a first penalty for being changed to another hydrophobic amino acid and a second, different penalty for being changed to a positively charged amino acid. The position modification data 312 can include numerical values related to probabilities and/or penalties corresponding to the modification of individual amino acids included in at least one of the one or more base sequences 306 or the one or more grafting sequences 310. To illustrate, the position modification data 312 can include numerical values from 0 to 1, numerical values from −1 to 1, and/or values from 0 to 100.

In one or more examples, the position modification data 312 can be determined, at least in part, based on input obtained via a computing device. For example, a user interface can be generated that includes one or more user interface elements to capture at least a portion of the position modification data 312. In addition, a data file can be obtained over a communication interface that includes at least a portion of the position modification data 312. Further, the position modification data 312 can be computed by analyzing a number of amino acid sequences to determine numbers of occurrences of different amino acids at one or more positions of the proteins. Occurrences of amino acids at positions of proteins, including base proteins and variant proteins, can be used to determine probabilities of modifications of amino acids that are indicated in the position modification data 312. In various examples, biophysical properties and/or structural features of proteins can be analyzed in conjunction with the placement of amino acids at one or more positions of base proteins and variant proteins to determine probabilities included in the position modification data 312 for modifying amino acids at one or more positions of base proteins to generate variant proteins.

The protein sequence combining component 314 can combine one or more portions of at least one base sequence 106 and one or more portions of at least one grafting sequence 310 according to the position modification data 312. The protein sequence combining component 314 can combine a portion of a base sequence 306 and a portion of a grafting sequence 310 by substituting amino acids located at one or more positions of a base sequence 306 with one or more amino acid located at one or more positions of a grafting sequence 310. The position modification data 312 can designate one or more positions of the base sequence 306 that are to be replaced and one or more positions of the grafting sequence 310 that supply the amino acids to replace the amino acids located at the one or more positions of the base sequence 306. In one or more examples, the position modification data 312 can indicate positions of one or more grafting sequences 310 that correspond to positions of one or more base sequences 306. In this way, the protein sequence combining component 314 can determine the positions of a grafting sequence 310 that are to supply amino acids to respective positions of a base sequence 306.

The protein sequence combining component 314 can generate combined protein sequence data 316 by replacing one or more amino acids of a base sequence 306 with one or more amino acids of a grafting sequence 310. The combined protein sequence data 316 can include combined sequences 318. The combined sequences 318 can include amino acid sequences that include at least one position of a base sequence 306 that has been modified based on at least one amino acid of one or more grafting sequences 310.

The protein sequence combining component 314 can provide the combined protein sequence data 316 to the neural network component 106. The neural network component 106 can analyze one or more combined sequences 318 included in the combined protein sequence data 316. In one or more examples, the neural network component 106 can generate one or more loss values 320 for one or more combined sequences 318. The one or more loss values 320 can be generated by the neural network component 106 based on the training data 302. In various examples, the neural network component 106 can determine the loss values 320 by analyzing individual positions of the combined sequences 318 and determining loss values 320 for the individual positions of the combined sequences 318. The neural network component 106 can aggregate loss values 320 of the individual positions of a combined sequence 318 to determine a cumulative loss value 320 of the combined sequence 318. In one or more illustrative examples, the loss values 320 for given positions of a combined sequence 318 can indicate an amount of difference with respect to corresponding positions of amino acid sequences included in the training data 302. In one or more additional examples, the loss values 320 for a combined sequence 318 can be determined with respect to amino acid sequences included in the training data 302 that have at least one of one or more biophysical properties or one or more structural features. For example, the neural network component 106 can analyze the combined sequences 318 in relation to proteins having amino acid sequences included in the training data 302 that have a specified range of pH values or a specified range of solubility measurements in water at one or more temperatures. Further, the neural network component 106 can analyze the combined sequences 318 in relation to amino acid sequences included in the training data 302 that have at least a threshold number of negatively charged amino acids in one or more regions.

In one or more examples, the loss values 320 can be provided to the variant generating component 108. The variant generating component 108 can utilize the loss values 320 in conjunction with the combined sequences 318 to generate variant sequence data 322. The variant sequence data 322 can include one or more variant sequences 324. The variant generating component 108 can generate the one or more variant sequences 324 by modifying amino acids located at one or more positions of the combined sequences 318. In various examples, the variant generating component 108 can determine positions of a combined sequence 318 that have loss values 320 that satisfy one or more criteria. For example, the variant generating component 108 can determine one or more positions of a combined sequence 318 that have at least a threshold loss value. In one or more illustrative examples, the threshold loss value can be a minimum loss value. In one or more additional examples, the variant generating component 108 can generate a ranked list of positions of a combined sequence 318 and determine one or more positions having the highest loss values 320, such as three positions having the highest loss values 320, five positions having the highest loss values 320, or ten positions having the highest loss values 320.

The variant generating component 108 can modify at least a portion of the one or more positions of the combined sequence 318 that satisfy the one or more criteria. To illustrate, the variant generating component 108 can generate a variant sequence 324 by modifying amino acids located at positions of a base sequence 306 to reduce the respective loss values 320 associated with the positions. In one or more examples, the variant generating component 318 can generate a variant sequence 324 by modifying amino acids located at positions of a combined sequence 306 to minimize the respective loss values 320 associated with the positions. In one or more additional examples, the variant generating component 318 can generate a variant sequence 324 by modifying amino acids located at positions of the combined sequence 318 to minimize an overall loss value 320 of the combined sequence 318.

In various examples, the position modification data 312 can be used by the variant generating component 108 to determine amino acids of a combined sequence 318 to modify to generate a variant sequence 324. For example, in scenarios where a combined sequence 318 includes one or more amino acids of a base sequence 306 and/or one or more amino acids of a grafting sequence 310 for which position modification data 312 is available, the variant generating component 108 can analyze the position modification data 312 for the respective positions to determine whether or not the amino acids located at the respective positions are to be modified. In one or more examples, the variant generating component 108 can analyze one or more loss values 320 for a combined sequence 318 in conjunction with position modification data 312 to determine one or more amino acids of the combined sequence 318 to modify in order to generate a variant sequence 324. To illustrate, a position of a combined sequence 318 can have a loss value 320 and a modification penalty indicated by the position modification data 312. In one or more illustrative examples, the loss value 320 and the modification penalty can be weighted and the variant generating component 108 can analyze a magnitude of the loss value 320 and a magnitude of the modification penalty of the position in relation to a weighting of the loss value 320 and a weighting of the modification penalty to determine whether to modify the amino acid located at the position. In an example scenario, a loss value 320 for an amino acid located at a position of a combined sequence 318 can be relatively high while a modification penalty for the amino acid can also be relatively high. In this situation, the variant generating component 108 can refrain from modifying the amino acid location at the position. In another example scenario, the loss value 320 for an amino acid located at a position of a combined sequence 318 can be relatively high while a modification penalty for the amino acid can be moderate. In this instance, the variant generating component 108 can determine that a reduction in the loss value 320 outweighs the modification penalty and the variant generating component 108 can modify the amino acid located at the position of the combined sequence 318.

The variant generating component 108 can modify one or more amino acids of a combined sequence 318 to generate a corresponding variant sequence 324 that has at least one of one or more biophysical properties or one or more structural features different from the initial combined sequence 318. For example, the variant generating component 108 can determine one or more amino acids of a combined sequence 318 to modify to generate a variant sequence 324 that has a value of a biophysical property, such as pH or percentage of high molecular weight segments, that is greater than the value of the biophysical property for the initial combined sequence 318. In one or more additional examples, the variant generating component 108 can determine one or more amino acids of a combined sequence to modify to generate a variant sequence 324 that has a greater number of regions having at least a minimum number of hydrophobic amino acids than the initial combined sequence 318.

In one or more additional examples, the protein sequence generating system 102 can generate scores for one or more combined sequences 318. For example, the protein sequence generating system 102 can generate scores for one or more combined sequences 318 based on the loss values 320. To illustrate, the protein sequence generating system 102 can generate a score for a combined sequence 318 based on an overall loss score for the combined sequence 318. In one or more examples, a score generated by the protein sequence generating system 102 can have a greater value when loss scores 320 for combined sequences 318 are lower. In these scenarios, the more that a combined sequence 318 corresponds to amino acid sequences included in the training data 302, the higher the score and the lower the loss values 320. In this way, the protein sequence generating system 102 can determine combined sequences 318 having values of desired biophysical properties and/or structural features that satisfy one or more criteria based on the scores of the combined sequences 318 generated by the protein sequence generating system 102 based on the loss values 320. In one or more further examples, the protein sequence generating system 102 can generate scores of combined sequences 318 having lower values when loss scores 320 for the combined sequences 318 are higher.

In the illustrative example of FIG. 3, a first base sequence 326, a second base sequence 328, and a third base sequence 330 can be combined by the protein sequence generating system 102 with a grafting sequence 332. In one or more examples, the base sequences 326, 328, 330 can be amino acid sequences of proteins produced by humans and the grafting sequence 310 can be an amino acid sequence of a protein produced by a mammal that is not a human. For example, the grafting sequence 332 can be an amino acid sequence of a protein produced by a mouse. In one or more illustrative examples, the base sequences 326, 328, 330 can be amino acid sequences of complementarity determining regions of one or more antibodies produced by humans and the grafting sequence 332 can be an amino acid sequence of a complementarity determining region of an antibody produced by a non-human mammal.

The base sequences 326, 328, 330 can have one or more regions that are to be modified based on the grafting sequence 332. For example, the first base sequence 326 can include a first region 334, a second region 336, and a third region 338 that are to be modified based on the grafting sequence 332. In various examples, the grafting sequence 332 can include a fourth region 340, a fifth region 342, and a sixth region 344 that include amino acids that can be used to modify the base sequences 326, 328, 330. In one or more examples, the fourth region 340, the fifth region 342, and the sixth region 344 can include a same number of amino acids as the regions to be modified in the base sequences 326, 328, 330. To illustrate, the fourth region 340 can include a same number of amino acids as the first region 334, the fifth region 342 can include a same number of amino acids as the second region 336, and the sixth region 344 can include a same number of amino acids as the third region 338. In this way, the first region 334 can be replaced by the fourth region 340, the second region 336 can be replaced by the fifth region 342, and the third region 338 can be replaced by the sixth region 344.

In one or more additional examples, at least a portion of the amino acids included in the fourth region 340, the fifth region 342, and the sixth region 344 can replace at least a portion of the amino acids included in regions of the base sequences 326, 328, 330. For example, the protein sequence combining component 314 can determine that at least a portion of the amino acids included in the fourth region 340, the fifth region 342, and/or the sixth region 344 can are to replace one or more amino acids included in at least one of the first region 334, the second region 336, or the third region 338.

The protein sequence combining component 314 can modify one or more amino acids included in one or more regions of the first base sequence 326 with one or more amino acids included in one or more regions of the grafting sequence 332 to generate a first combined sequence 346. The protein sequence combining component 314 can also modify one or more amino acids included in one or more regions of the second base sequence 328 with amino acids included in one or more regions of the grafting sequence 332 to generate a second combined sequence 348. In addition, the protein sequence combining component 314 can modify one or more amino acids included in one or more regions of the third base sequence 330 with one or more amino acids included in one or more regions of the grafting sequence 332 to generate a third combined sequence 350. In various examples, the regions of the first base sequence 326 that are modified in accordance with the regions of the grafting sequence 332 can be different from the regions of at least one of the second base sequence 328 or the third base sequence 330 that are modified in accordance with the regions of the grafting sequence 332. Additionally, the regions of the second base sequence 328 that are modified in accordance with regions of the grafting sequence 332 can be different from the regions of the third base sequence 330 that are modified in accordance with the grafting sequence 332. Further, in one or more examples, different regions of the grafting sequence 332 can be used to replace different regions of amino acids of the base sequences 326, 328, 330. For example, the fourth region 340 of the grafting sequence 332 can replace the first region 334 of the first base sequence 326 and a different region of the grafting sequence 332 can replace an additional region of the second base sequence 328 that does not correspond to the first region 326. In one or more illustrative examples, the base sequences 326, 328, 330 can correspond to heavy chain regions of antibodies with the first base sequence 326 corresponding to positions 1-18 of a heavy chain region, the second base sequence 328 corresponding to positions 3-30 of a heavy chain region, and the third base sequence 330 corresponding to positions 4-39 of a heavy chain region.

The protein sequence generating system 102 can, at operation 352, evaluate the combined sequences 346, 348, 350. For example, the protein sequence generating system 102 can determine a first score for the first combined sequence 346, a second score for the second combined sequence 348, and a third score for the third combined sequence 350. The protein sequence generating system 102 can evaluate the first score, the second score, and the third score to determine a combined sequence 346, 348, 350 having at least one of a desired biophysical property or a desired structural feature. In one or more examples, the protein sequence generating system 102 can determine a combined sequence 346, 348, 350 having a highest score. In the illustrative example of FIG. 3, the protein sequence generating system 102 can determine that the third combined sequence 350 has a highest score in relation t at least one of the desired biophysical property or the desired structural feature.

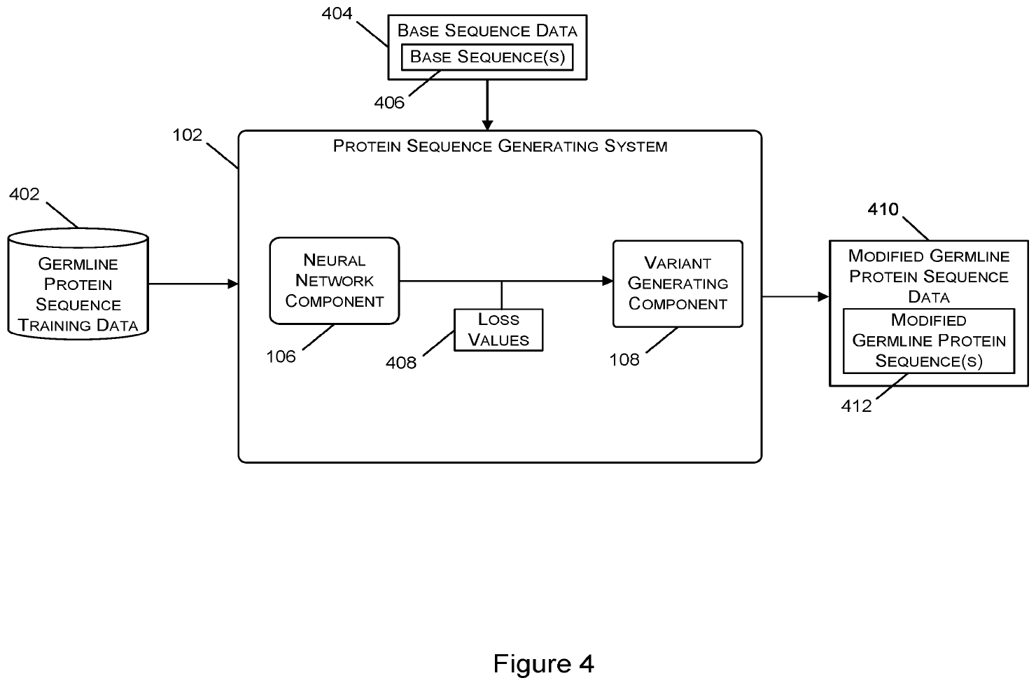

FIG. 4 is a diagram illustrating an example framework 400 to generate protein sequences directed to a respective germline using a neural network component, in accordance with one or more implementations. The framework 400 includes the protein sequence generating system 102. The protein sequence generating system 102 can include the neural network component 106 and the variant generating component 108. The neural network component 106 can be trained using germline protein sequence training data 402. The germline protein sequence training data 402 can include amino acid sequences of proteins that are produced in accordance with genomic regions of one or more germlines. In one or more examples, the neural network component 106 can be trained using the germline sequence training data 402 to identify amino acid sequences that correspond to the amino acid sequences included in the germline sequence training data 402. The neural network component 106 can also be trained using the germline protein sequence training data 402 to generate amino acid sequences that correspond to the amino acid sequences included in the germline protein sequence training data 402. Further, the neural network component 106 can be trained using the germline protein sequence training data 402 to generate amino acid sequences of proteins having one or more characteristics of proteins and/or protein fragments corresponding to the amino acid sequences included in the germline protein sequence training data 402. In one or more illustrative examples, the neural network component 106 can include one or more convolutional neural networks. In these scenarios, the training data 402 can be used to train one or more kernels of the one or more convolutional neural networks.

The amino acid sequences included in the germline protein sequence training data 402 can correspond to at least one of proteins or protein fragments generated in accordance with one or more human germline genomic regions. In one or more examples, the germline protein sequence training data 402 can include amino acid sequences of at least one of antibodies or antibody sequences generated in accordance with one or more human germline genomic regions. In one or more illustrative examples, the training data 402 can include amino acid sequences of fibronectin type III (FNIII) proteins, avimers, VHH domains, kinases, zinc fingers, T-cell receptors, one or more combinations thereof, and the like, generated in accordance with one or more human germline genomic regions.

The protein sequence generating system 102 can obtain base sequence data 404. The base sequence data 404 can include one or more base sequences 406. The one or more base sequences 406 can correspond to amino acid sequences of proteins that are to be analyzed by the protein sequence generating system 102. In one or more examples, the base sequence data 404 can include base sequences 406 that correspond to amino acid sequences of at least one of proteins or protein fragments generated in accordance with one or more germline genomic regions that are different from the germline genomic regions used to produce the germline protein sequence training data 402. For example, the amino acid sequences included in the germline protein sequence training data 402 can correspond to proteins generated in accordance with one or more first germline genomic regions and the base sequences 406 can correspond to proteins generated according to one or more second germline genomic regions. In one or more illustrative examples, the one or more first germline genomic regions related to the germline protein sequence training data 402 can include first human germline genomic regions and the one or more second germline genomic regions related to the base sequences 406 can include second human germline genomic regions. In one or more additional illustrative examples, the one or more first germline genomic regions related to the germline protein sequence training data 402 can include human germline genomic regions and the one or more second germline genomic regions related to the base sequences 406 can include non-human germline genomic regions. In these scenarios, the one or more second germline genomic regions can correspond to germline genomic regions of a non-human mammal.

The neural network component 106 can analyze the one or more base sequences 406 included in the base sequence data 406. In one or more examples, the neural network component 106 can generate one or more loss values 408 for one or more base sequences 406. The one or more loss values 408 can be generated by the neural network component 106 based on the germline protein sequence training data 402. In various examples, the neural network component 106 can determine the loss values 408 by analyzing individual positions of the base sequences 406 and determining loss values 408 for the individual positions of the base sequences 406. The neural network component 106 can aggregate loss values 408 of the individual positions of a base sequence 406 to determine a cumulative loss value 408 of the base sequence 406. In one or more illustrative examples, the loss values 408 for given positions of a base sequence 406 can indicate an amount of difference with respect to corresponding positions of amino acid sequences included in the germline protein sequence training data 402. In various examples, the loss values 408 can indicate a measure of similarity or a measure of correspondence between one or more positions of the base sequences 406 with respect to one or more positions of the amino acid sequences included in the germline protein sequence training data 402.

The loss values 408 can be provided to the variant generating component 108. The variant generating component 108 can utilize the loss values 408 to generate modified germline protein sequence data 410. The modified germline protein sequence data 410 can include one or more modified germline protein sequences 412. In one or more examples, the modified germline protein sequences 412 can be variants of the base sequences 406 that have one or more characteristics that are more similar with respect to the germline protein sequence training data 402 than the base sequences 406. The variant generating component 108 can generate the one or more modified germline protein sequences 412 by modifying amino acids located at one or more positions of the base sequences 406. In various examples, the variant generating component 108 can determine positions of a base sequence 406 that have loss values 408 that satisfy one or more criteria. For example, the variant generating component 108 can determine one or more positions of a base sequence 406 that have at least a threshold loss value. In one or more illustrative examples, the threshold loss value can be a minimum loss value. In one or more additional examples, the variant generating component 108 can generate a ranked list of positions of a base sequence 406 and determine one or more positions having the highest loss values 408.

The variant generating component 108 can modify at least a portion of the one or more positions of the base sequences 406 that satisfy the one or more criteria. To illustrate, the variant generating component 108 can generate a modified germline sequence 412 by modifying amino acids located at positions of a base sequence 406 to reduce the respective loss values 408 associated with the positions. In one or more examples, the variant generating component 108 can generate a modified germline protein sequence 412 by modifying amino acids located at positions of a base sequence 406 to minimize the respective loss values 408 associated with the positions. In one or more additional examples, the variant generating component 108 can generate a modified germline protein sequence 412 by modifying amino acids located at positions of the base sequence 406 to minimize an overall loss value 408 of the base sequence 406.

The variant generating component 108 can modify one or more amino acids of a base sequence 406 to generate a corresponding modified germline protein sequence 412 that has at least one of one or more biophysical properties or one or more structural features different from the initial base sequence 406. In one or more examples, the modifications made to a base sequence 406 by the variant generating component 108 to produce a corresponding modified germline protein sequence 412 can result in the modified germline protein sequence 412 having a greater similarity of characteristics of proteins produced from germline genomic regions used to produce the amino acid sequences included in the germline protein sequence training data 402 in relation to characteristics of proteins produced in accordance with germline genomic regions used to produce the base sequences 406. In one or more illustrative examples, the germline protein sequence training data 402 can correspond to amino acid sequences of proteins produced in accordance with one or more human germline genomic regions and the base sequences 406 can correspond to amino acid sequences of proteins produced in accordance with one or more non-human mammal germline genomic regions. Continuing with this example, the variant generating component 108 can produce modified germline protein sequences 412 from the base sequences 406 that are more similar to amino acid sequences of proteins produced in accordance with the one or more human germline genomic regions than amino acid sequences of proteins produced in accordance with the non-human mammal germline genomic regions. In this way, the variant generating component 108 can modify amino acids of the base sequences 406 to transform the base sequences 406 to be more similar to amino acid sequences of proteins produced according to one or more human germline genomic regions. In one or more further illustrative examples, the base sequences 406 can correspond to amino acid sequences of proteins produced in accordance with different human germline genomic regions than the amino acid sequences of proteins included in the germline protein sequence training data 402. In these scenarios, the variant generating component 108 can modify amino acids of the base sequences 406 to transform the base sequences 406 to be more similar to amino acid sequences of proteins produced by the human germline genomic regions related to the germline protein sequence data 402 than proteins produced according to the different human germline genomic regions related to the base sequences 406.

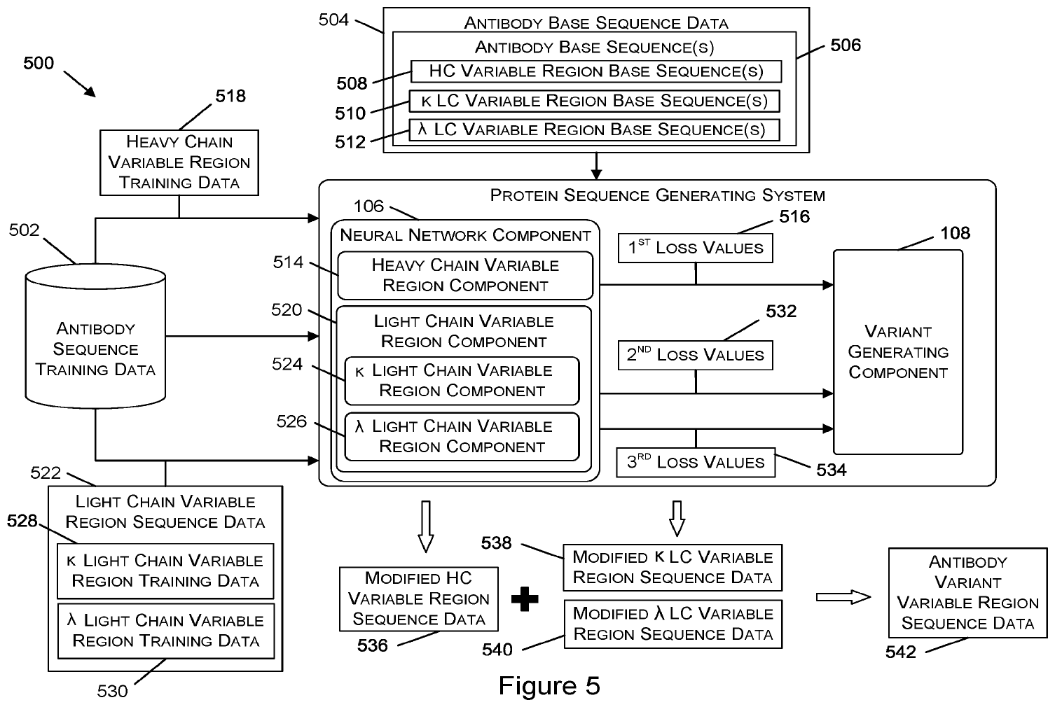

FIG. 5 is a diagram illustrating an example framework 500 to generate sequences of antibodies using a neural network component, in accordance with one or more implementations. The framework 500 includes the protein sequence generating system 102. The protein sequence generating system 102 can include the neural network component 106 and the variant generating component 108. The neural network component 106 can be trained using antibody sequence training data 502. In one or more examples, the neural network component 106 can be trained using the antibody sequence training data 502 to identify amino acid sequences that correspond to the amino acid sequences included in the antibody sequence training data 502. The neural network component 106 can also be trained using the antibody sequence training data 502 to generate amino acid sequences that correspond to the amino acid sequences included in the antibody sequence training data 502. Further, the neural network component 106 can be trained using the antibody sequence training data 502 to generate amino acid sequences of antibodies having one or more characteristics of antibodies and/or antibody fragments corresponding to the amino acid sequences included in the antibody sequence training date 502. In one or more illustrative examples, the neural network component 106 can include one or more convolutional neural networks. In these scenarios, the antibody sequence training data 502 can be used to train one or more kernels of the one or more convolutional neural networks.

The amino acid sequences included in the antibody sequence training data 502 can correspond to at least one of antibodies or antibody fragments generated by humans. In various examples, the amino acid sequences included in the antibody sequence training data 502 can have characteristics of at least one of antibodies or antibody fragments generated by humans. Additionally, the antibody sequence training data 502 can include amino acid sequences of at least one of antibodies or antibody fragments produced in accordance with one or more human germline genomic regions. In various examples, the antibody sequence training data 502 can include amino acid sequences of at least one of antibody heavy chains or antibody light chains. In one or more illustrative examples, the antibody sequence training data 502 can include amino acid sequences of at least one of antibody light chain variable regions or antibody heavy chain variable regions. In one or more additional illustrative examples, the antibody sequence training data 502 can include amino acid sequences of at least one of complementarity determining regions of antibody light chains or complementarity determining regions of antibody heavy chain regions. In one or more further illustrative examples, the antibody sequence training data 502 can include amino acid sequences of at least one of kappa antibody light chains or lambda antibody light chains.

The protein sequence generating system 102 can obtain antibody base sequence data 504. The antibody base sequence data 504 can include one or more antibody base sequences 506. The one or more antibody base sequences 506 can correspond to amino acid sequences of antibodies and/or antibody fragments that are to be analyzed by the protein sequence generating system 102. In one or more illustrative examples, the antibody base sequences 506 can include one or more heavy chain (HC) variable region base sequences 508, one or more kappa light chain (LC) variable region base sequences 510, and one or more lambda light chain (LC) variable region base sequences 512. In various examples, the protein sequence generating system 102 can generate variant antibody sequences based on the antibody base sequences 506. In one or more examples, the antibody base sequence data 504 can include antibody base sequences 506 that correspond to amino acid sequences of at least one of antibodies or antibody fragments generated in accordance with one or more human genomic regions. In one or more additional examples, the antibody base sequence data 504 can include antibody base sequences 506 that correspond to amino acid sequences of at least one of antibodies or antibody fragments generated in accordance with one or more non-human mammalian genomic regions. In these scenarios, the protein sequence generating component 102 can generate variant sequences that are humanized in relation to the antibody base sequences 506.

In one or more examples, an amino acid sequence of an antibody or antibody fragment can be humanized by modifying a portion of the amino acids included in an initial amino acid sequence to produce a modified amino acid sequence such that the modified amino acid sequence has characteristics that are more similar to amino acid sequences produced in accordance with human genomic regions than the initial amino acid sequence. A modified amino acid sequence can be more similar to amino acid sequences produced in accordance with human genomic regions by modifying amino acids located at one or more regions of the initial amino acid sequence to have a greater amount of homology with respect to one or more regions of amino acid sequences of proteins produced in accordance with human genomic regions. Further, a modified amino acid sequence can correspond to a variant antibody that is more similar to human antibodies than a base antibody that corresponds to the initial amino acid sequence based on at least one of one or more biophysical properties or one or more structural features of the variant antibody being more similar to human antibodies than the base antibody.

The neural network component 106 can include a heavy chain variable region component 514. The heavy chain variable region component 514 can identify at least one of heavy chain variable regions of antibodies or a portion of heavy chain variable regions of antibodies for analysis. In one or more examples, the heavy chain variable region component 514 can analyze the heavy chain variable region base sequences 508. In one or more examples, the heavy chain variable region component 514 can implement one or more models to identify and analyze at least portions of heavy chain variable regions of antibodies.

The heavy chain variable region component 514 can generate one or more first loss values 516 by analyzing the heavy chain variable region base sequences 508. The one or more first loss values 516 can be generated by the heavy chain variable region component 514 based on the antibody sequence training data 502. In various examples, the antibody sequence training data 502 can include heave chain variable region training data 518 and the heavy chain variable region component 508 can determine the first loss values 516 by analyzing individual positions of the HC variable region base sequences 508 with respect to corresponding positions of the heavy chain variable region training data 518. The heavy chain variable region component 514 can then determine the first loss values 516 for the individual positions of the HC variable region base sequences 508. The heavy chain variable region component 514 can aggregate the first loss values 516 of the individual positions of a heavy chain variable region base sequence 508 to determine a cumulative first loss value 516 of the heavy chain variable region base sequence 508. In one or more illustrative examples, the first loss values 516 for given positions of a heavy chain variable region base sequence 508 can indicate an amount of difference with respect to corresponding positions of amino acid sequences included in the heavy chain variable region training data 518. In one or more additional examples, the first loss values 516 for a heavy chain variable region base sequence 508 can be determined with respect to amino acid sequences included in the heavy chain variable region training data 518 that have at least one of one or more biophysical properties or one or more structural features. For example, the heavy chain variable region component 514 can analyze the heavy chain variable region base sequences 508 in relation to antibodies having amino acid sequences included in the heavy chain variable region training data 518 that have a specified range of pH values or a specified range of solubility measurements in water at one or more temperatures to determine the first loss values 516. Further, the heavy chain variable region component 514 can analyze the heavy chain variable region base sequences 508 in relation to amino acid sequences included in the heavy chain variable region training data 518 that have at least a threshold number of negatively charged amino acids in one or more regions to determine the first loss values 516.

The neural network component 106 can also include a light chain variable region component 520. The light chain variable region component 520 can identify at least one of light chain variable regions of antibodies or a portion of light chain variable regions of antibodies for analysis. In one or more examples, the light chain variable region component 520 can analyze light chain variable region sequence data 522 included in the antibody sequence training data 502 with respect to light chain variable region base sequences included in the antibody base sequence data 506. In various examples, the light chain variable region component 520 can analyze light chain variable region base sequences with respect to the light chain variable region training data 522 to determine loss values for the light chain variable region base sequences. In one or more examples, the light chain variable region component 520 can implement one or more models to identify and analyze at least portions of light chain variable regions of antibodies.

In various examples, the light chain variable region component 520 can include individual components to analyze different antibody types, such as antibodies having kappa light chains and antibodies having lambda light chains. For example, the light chain variable region component 520 can include a kappa light chain variable region component 524 and a lambda light chain variable region component 526. The kappa light chain variable region component 524 can identify and analyze kappa light chain variable region base sequences 510 in relation to kappa light chain region training data 528. In addition, the lambda light chain variable region component 526 can identify and analyze lambda light chain variable region base sequences 512 in relation to lambda light chain region training data 530. In one or more examples, the kappa light chain variable region component 524 can implement one or more first models to identify and analyze at least portions of kappa light chain variable regions of antibodies and the lambda light chain variable region component 526 can implement one or more second models to identify and analyze at least portions of lambda light chain variable regions of antibodies.

The kappa light chain variable region component 524 can generate one or more second loss values 532 by analyzing the kappa light chain variable region base sequences 510. The one or more second loss values 532 can be generated by the kappa light chain variable region component 524 based on the kappa light chain variable region training data 528. In various examples, the kappa light chain variable region component 524 can determine the second loss values 532 by analyzing individual positions of the kappa light chain variable region base sequences 510 with respect to corresponding positions of the kappa light chain variable region training data 528. The kappa light chain variable region component 524 can then determine the second loss values 532 for the individual positions of the kappa light chain variable region base sequences 510. The kappa light chain variable region component 524 can aggregate the second loss values 532 of the individual positions of a kappa light chain variable region base sequence 510 to determine a cumulative second loss value of the kappa light chain variable region base sequence 510. In one or more illustrative examples, the second loss values 532 for given positions of a kappa light chain variable region base sequence 510 can indicate an amount of difference with respect to corresponding positions of amino acid sequences included in the kappa light chain variable region training data 528. In one or more additional examples, the second loss values 532 for a kappa light chain variable region base sequence 510 can be determined with respect to amino acid sequences included in the kappa light chain variable region training data 528 that have at least one of one or more specified biophysical properties or one or more specified structural features.

The lambda light chain variable region component 526 can generate one or more third loss values 534 by analyzing the lambda light chain variable region base sequences 512. The one or more third loss values 534 can be generated by the lambda light chain variable region component 526 based on the lambda light chain variable region training data 530. In various examples, the lambda light chain variable region component 526 can determine the third loss values 534 by analyzing individual positions of the lambda light variable region base sequences 512 with respect to corresponding positions of the lambda light chain variable region training data 530. The lambda light chain variable region component 524 can then determine the third loss values 534 for the individual positions of the lambda light chain variable region base sequences 512. The lambda light chain variable region component 526 can aggregate the third loss values 534 of the individual positions of a lambda light chain variable region base sequence 512 to determine a cumulative loss value of the lambda light chain variable region base sequence 512. In one or more illustrative examples, the third loss values 534 for given positions of a lambda light chain variable region base sequence 512 can indicate an amount of difference with respect to corresponding positions of amino acid sequences included in the lambda light chain variable region training data 530. In one or more additional examples, the third loss values 534 for a lambda light chain variable region base sequence 512 can be determined with respect to amino acid sequences included in the lambda light chain variable region training data 530 that have at least one of one or more specified biophysical properties or one or more specified structural features.

In one or more examples, the first loss values 516 can be provided to the variant generating component 108. The variant generating component 108 can utilize the first loss values 516 in conjunction with the heavy chain variable region base sequences 508 to generate modified heavy chain variable region sequence data 536. The modified heavy chain variable region sequence data 536 can include heavy chain variable region sequences that can be used to produce variant antibody sequences. The variant generating component 108 can generate the modified heavy chain variable region sequence data 536 by modifying amino acids located at one or more positions of the heavy chain variable region base sequences 508. In various examples, the variant generating component 108 can determine positions of a heavy chain variable region base sequence 508 that have first loss values 516 that satisfy one or more criteria. For example, the variant generating component 108 can determine one or more positions of a heavy chain variable region base sequence 508 that have at least a threshold loss value. In one or more illustrative examples, the threshold loss value can be a minimum loss value. In one or more additional examples, the variant generating component 108 can generate a ranked list of positions of a heavy chain variable region base sequence 508 and determine one or more positions having the highest first loss values 516.

The variant generating component 108 can modify at least a portion of the one or more positions of the heavy chain variable region base sequence 508 that satisfy the one or more criteria. To illustrate, the variant generating component 108 can generate the modified heavy chain variable region sequence data 536 by modifying amino acids located at positions of a heavy chain variable region base sequence 508 to reduce the respective first loss values 516 associated with the positions. In one or more examples, the variant generating component 108 can generate the modified heavy chain variable region sequence data 536 by modifying amino acids located at positions of a heavy chain variable region base sequence 508 to minimize the respective first loss values 516 associated with the positions. In one or more additional examples, the variant generating component 108 can generate the modified heavy chain variable region sequence data 536 by modifying amino acids located at positions of the heavy chain variable region base sequence 508 to minimize an overall loss value of the heavy chain variable region base sequence 508. The variant generating component 108 can modify one or more amino acids of a heavy chain variable region base sequence 508 to generate a corresponding heavy chain variable region variant sequence that has at least one of one or more biophysical properties or one or more structural features different from the initial heavy chain variable region base sequence 508.

In one or more implementations, the second loss values 532 can be provided to the variant generating component 108. The variant generating component 108 can utilize the second loss values 532 in conjunction with the kappa light chain variable region base sequences 510 to generate modified kappa light chain variable region sequence data 538. The modified kappa light chain variable region sequence data 538 can include kappa light chain variable region sequences that can be used to produce variant antibody sequences. The variant generating component 108 can generate the modified kappa light chain variable region sequence data 538 by modifying amino acids located at one or more positions of the kappa light chain variable region base sequences 510. In various examples, the variant generating component 108 can determine positions of a kappa light chain variable region base sequence 510 that have second loss values 532 that satisfy one or more criteria. For example, the variant generating component 108 can determine one or more positions of a kappa light chain variable region base sequence 510 that have at least a threshold loss value. In one or more illustrative examples, the threshold loss value can be a minimum loss value. In one or more additional examples, the variant generating component 108 can generate a ranked list of positions of a kappa light chain variable region base sequence 510 and determine one or more positions having the highest second loss values 532.

The variant generating component 108 can modify at least a portion of the one or more positions of the kappa light chain variable region base sequence 510 that satisfy the one or more criteria. To illustrate, the variant generating component 108 can generate the modified kappa light chain variable region sequence data 538 by modifying amino acids located at positions of a kappa light chain variable region base sequence 510 to reduce the respective first second values 532 associated with the positions. In one or more examples, the variant generating component 108 can generate the modified kappa light chain variable region sequence data 538 by modifying amino acids located at positions of a kappa light chain variable region base sequence 510 to minimize the respective second loss values 532 associated with the positions. In one or more additional examples, the variant generating component 108 can generate the modified kappa light chain variable region sequence data 538 by modifying amino acids located at positions of the kappa light chain variable region base sequence 510 to minimize an overall loss value of the kappa light chain variable region base sequence 510. The variant generating component 108 can modify one or more amino acids of a kappa light chain variable region base sequence 510 to generate a corresponding kappa light chain variable region variant sequence that has at least one of one or more biophysical properties or one or more structural features different from the initial kappa light chain variable region base sequence 510.

In various examples, the third loss values 534 can be provided to the variant generating component 108. The variant generating component 108 can utilize the third loss values 534 in conjunction with the lambda light chain variable region base sequences 512 to generate modified lambda light chain variable region sequence data 540. The modified lambda light chain variable region sequence data 540 can include lambda light chain variable region sequences that can be used to produce variant antibody sequences. The variant generating component 108 can generate the modified lambda light chain variable region sequence data 540 by modifying amino acids located at one or more positions of the lambda light chain variable region base sequences 512. In various examples, the variant generating component 108 can determine positions of a lambda light chain variable region base sequence 512 that have third loss values 534 that satisfy one or more criteria. For example, the variant generating component 108 can determine one or more positions of a lambda light chain variable region base sequence 512 that have at least a threshold loss value. In one or more illustrative examples, the threshold loss value can be a minimum loss value. In one or more additional examples, the variant generating component 108 can generate a ranked list of positions of a lambda light chain variable region base sequence 512 and determine one or more positions having the highest third loss values 534.

The variant generating component 108 can modify at least a portion of the one or more positions of the lambda light chain variable region base sequence 512 that satisfy the one or more criteria. To illustrate, the variant generating component 108 can generate the modified lambda light chain variable region sequence data 540 by modifying amino acids located at positions of a lambda light chain variable region base sequence 512 to reduce the respective third loss values 534 associated with the positions. In one or more examples, the variant generating component 108 can generate the modified lambda light chain variable region sequence data 540 by modifying amino acids located at positions of a lambda light chain variable region base sequence 512 to minimize the respective third loss values 534 associated with the positions. In one or more additional examples, the variant generating component 108 can generate the modified lambda light chain variable region sequence data 540 by modifying amino acids located at positions of the lambda light chain variable region base sequence 512 to minimize an overall loss value of the lambda light chain variable region base sequence 512. The variant generating component 108 can modify one or more amino acids of a lambda light chain variable region base sequence 512 to generate a corresponding lambda light chain variable region variant sequence that has at least one of one or more biophysical properties or one or more structural features different from the initial lambda light chain variable region base sequence 512.

In the illustrative example of FIG. 5, the protein sequence generating system 102 can combine modified heavy chain variable region sequence data 536 with modified kappa light chain variable region sequence data 538 or modified lambda light chain variable region sequence data 540 to generate antibody variant variable region sequence data 542. The antibody variant variable region sequence data 542 can include amino acid sequences of antibody variable regions that include a heavy chain variable region and a light chain variable region that have been modified in relation to the antibody base sequences 506. In one or more examples, the antibody base sequences 506 can be amino acid sequences of antibodies produced by non-human mammals and the protein sequence generating system 102 can humanize the antibody base sequences 506 based on heavy chain variable region training data 518 and light chain variable region training data 522 that include amino acid sequences of antibody variable regions produced in accordance with one or more human genomic regions. In various examples, the antibody variant variable region sequence data 542 can be combined with additional antibody sequence data to generate antibody sequences. For example, the antibody variant variable region sequence data 542 can be combined with constant region sequences, hinge region sequences, or a combination thereof to produce antibody sequences.

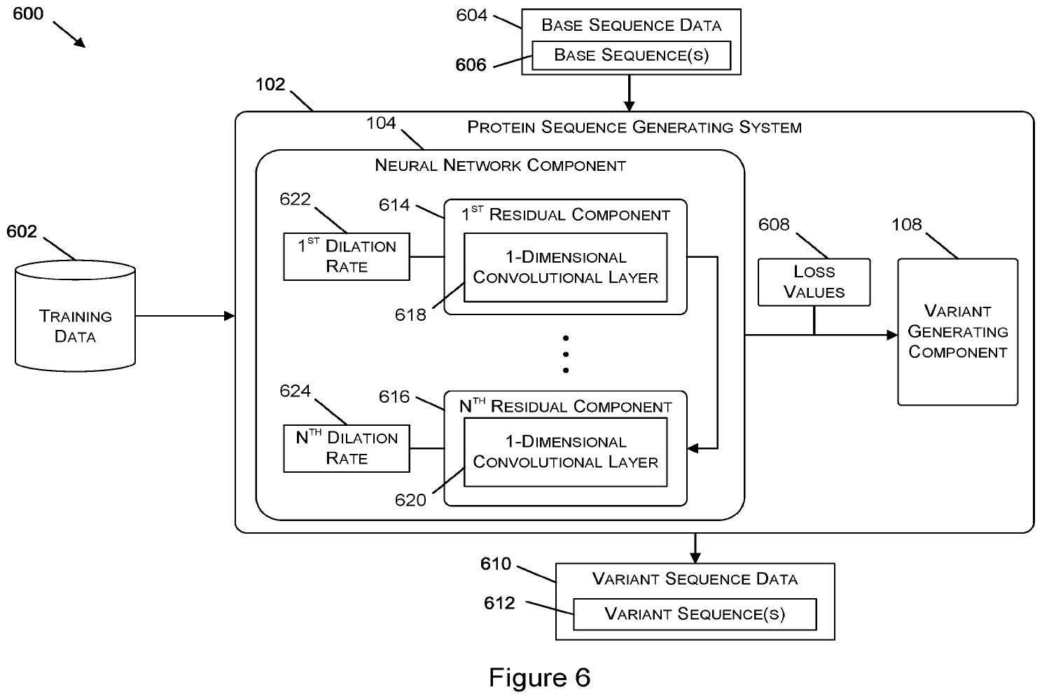

FIG. 6 is a diagram illustrating an example framework 600 to generate protein sequences using a residual neural network, in accordance with one or more implementations. The framework 600 includes the protein sequence generating system 102. The protein sequence generating system 102 can include the neural network component 106. The neural network component 106 can be trained using training data 602. The training data 602 can include amino acid sequences of proteins. In one or more examples, the neural network component 106 can be trained using the training data 602 to identify amino acid sequences that correspond to the amino acid sequences included in the training data 602. The neural network component 106 can also be trained using the training data 602 to generate amino acid sequences that correspond to the amino acid sequences included in the training data 602. Further, the neural network component 106 can be trained using the training data 602 to generate amino acid sequences of proteins having one or more characteristics of proteins and/or protein fragments corresponding to the amino acid sequences included in the training date 602. In one or more illustrative examples, the neural network component 106 can include one or more convolutional neural networks. In these scenarios, the training data 602 can be used to train one or more kernels of the one or more convolutional neural networks.

The amino acid sequences included in the training data 602 can correspond to at least one of proteins or protein fragments. In one or more examples, the amino acid sequences included in the training data 602 can have characteristics of at least one of proteins or protein fragments generated by humans. Additionally, the training data 602 can include amino acid sequences of at least one of proteins or protein fragments produced in accordance with one or more human germline genomic regions. In one or more illustrative examples, the training data 602 can include amino acid sequences of at least one of antibodies or antibody sequences. In various examples, the training data 602 can include amino acid sequences of at least one of antibodies or antibody sequences generated in accordance with one or more human germline genomic regions. In one or more additional illustrative examples, the training data 602 can include amino acid sequences of fibronectin type III (FNIII) proteins, avimers, VHH domains, kinases, zinc fingers, T-cell receptors, one or more combinations thereof, and the like.

The protein sequence generating system 102 can obtain base sequence data 604. The base sequence data 604 can include one or more base sequences 606. The one or more base sequences 606 can correspond to amino acid sequences of proteins that are to be analyzed by the protein sequence generating system 102. In one or more examples, the base sequence data 604 can include base sequences 606 that correspond to amino acid sequences of at least one of proteins or protein fragments generated in accordance with one or more human genomic regions. In one or more illustrative examples, the base sequence data 604 can include base sequences 606 that correspond to amino acid sequences of at least one of antibody or antibody fragments generated in accordance with one or more human genomic regions. In one or more additional illustrative examples, the base sequence data 604 can include base sequences 606 that correspond to amino acid sequences of fibronectin type III (FNIII) proteins, avimers, VHH domains, kinases, zinc fingers, T-cell receptors, one or more combinations thereof, and the like.

The neural network component 106 can analyze the one or more base sequences 606 and generate one or more loss values 608 for one or more base sequences 606. The one or more loss values 608 can be generated by the neural network component 106 based on the training data 602. In various examples, the neural network component 106 can determine the loss values 608 by analyzing individual positions of the base sequences 606 and determining loss values 608 for the individual positions of the base sequences 606. The neural network component 106 can aggregate loss values 606 of the individual positions of a base sequence 606 to determine a cumulative loss value of the base sequence 606. In one or more illustrative examples, the loss values 608 for given positions of a base sequence 606 can indicate an amount of difference with respect to corresponding positions of amino acid sequences included in the training data 602. In one or more additional examples, the loss values 608 for a base sequence 606 can be determined with respect to amino acid sequences included in the training data 602 that have at least one of one or more biophysical properties or one or more structural features.

In one or more examples, the loss values 608 can be provided to the variant generating component 108. The variant generating component 108 can utilize the loss values 608 in conjunction with the base sequences 606 to generate variant sequence data 610. The variant sequence data 610 can include one or more variant sequences 612. The variant generating component 108 can generate the one or more variant sequences 612 by modifying amino acids located at one or more positions of the base sequences 606. In various examples, the variant generating component 108 can determine positions of a base sequence 606 that have loss values 608 that satisfy one or more criteria. For example, the variant generating component 108 can determine one or more positions of a base sequence 606 that have at least a threshold loss value. In one or more illustrative examples, the threshold loss value can be a minimum loss value. In one or more additional examples, the variant generating component 108 can generate a ranked list of positions of a base sequence 606 and determine one or more positions having the highest loss values 608.

The variant generating component 108 can modify at least a portion of the one or more positions of the base sequence 606 that satisfy the one or more criteria. To illustrate, the variant generating component 108 can generate a variant sequence 612 by modifying amino acids located at positions of a base sequence 606 to reduce the respective loss values 608 associated with the positions. In one or more examples, the variant generating component 108 can generate a variant sequence 612 by modifying amino acids located at positions of a base sequence 606 to minimize the respective loss values 608 associated with the positions. In one or more additional examples, the variant generating component 108 can generate a variant sequence 612 by modifying amino acids located at positions of the base sequence 606 to minimize an overall loss value 608 of the base sequence 606.

The variant generating component 108 can modify one or more amino acids of a base sequence 606 to generate a corresponding variant sequence 612 that has at least one of one or more biophysical properties or one or more structural features different from the initial base sequence 606. For example, the variant generating component 108 can determine one or more amino acids of a base sequence 606 to modify to generate a variant sequence 612 that has a value of a biophysical property that is different than the value of the biophysical property for the initial base sequence 606.

In the illustrative example of FIG. 6, the neural network component 104 can include a number of residual components. For example, the neural network component 104 can include a first residual component 614 up to an Nth residual component 616. In one more examples, the residual components 614, 616 of the neural network component 104 can forward output of an activation layer to an additional residual component. To illustrate, the first residual component 614 can include an activation block that provides output of the activation block to a second residual component (not shown in FIG. 6) that is logically coupled to the first residual component 614. In various examples, the neural network component 104 can include a series of residual components with at least a portion of the output of individual residual components being carried forward to a subsequent residual component in the series of residual components.

The residual components 614, 616 can include a number of layers. For example, the first residual component 614 can include a first one-dimensional convolutional layer 618 and the Nth residual component 616 can include an Nth one-dimensional convolutional layer 620. In one or more examples, at least a portion of the first residual component 614 up to the Nth residual component 616 can include multiple one-dimensional convolutional layers. Additionally, the residual components 614, 616 can individually include one or more activation layers, one or more normalization layers, one or more regularization layers, or one or more combinations thereof. To illustrate, the residual components 614, 616 can individually include at least one batch normalization layer to normalize values of computations performed by the residual components 614, 616. In one or more examples, batch normalization layers can improve the efficiency of the neural network component 104 by causing one or more convolutional neural networks of the neural network component 104 to converge sooner than if the neural network component 104 was implemented without batch normalization layers. In one or more additional examples, the residual component 614, 616 can individually include at least one dilution layer, such as a dropout layer. The use of dilution layers in the residual components 614, 616 can reduce overfitting of data during the implementation of the neural network component 104.

In addition to the residual components 614, 616, the neural network component 104 can include at least one of one or more input layers or one or more embedding layers. The one or more input layers and/or the one or more embedding layers can modify input data according to one or more rules to enable the input data to be processed by the neural network component 104. Further, the neural network component 104 can also include at least one of one or more flattening layers or one or more fully connected layers. the neural network component 104 can also include one or more additional normalization layers, such as one or more layers that implement a softmax function.

In various examples, individual residual components, such as residual components 614, 616 can be implemented with respect to different dilation rates. For example, the first residual component 614 can be implemented in relation to a first dilation rate 622 and the Nth residual component 616 can be implemented in relation to an Nth dilation rate 624. The dilation rates 622, 624 can indicate defined gaps in the data being analyzed by the respective residual components 614, 616. In one or more illustrative examples, the first dilation rate 622 can indicate that a number of consecutive amino acids are analyzed by the first residual component 614 at a time, such as 2 consecutive amino acids, 3 consecutive amino acids, 4 consecutive amino acids, 5 consecutive amino acids, 7 consecutive amino acids, 10 consecutive amino acids. The number of amino acids analyzed can be based on a size of a kernel of one or more convolutional neural networks of the neural network component 104. The dilation rate can increase for additional residual components. To illustrate, a second residual component subsequent to the first residual component 614 can have a dilation rate of two indicating that for each position of an amino acid sequence being analyzed by the second residual component, a position of the amino acid sequence is skipped. Additionally, another residual component can have a dilation rate of four indicating that for each position of an amino acid sequence being analyzed, three positions of the amino acid sequence are skipped. In one or more illustrative examples, the Nth dilation rate 624 can be 4, 8, 16, 32, 64.

In one or more illustrative examples, a base sequence 606 can be provided to the neural network component 104 and data corresponding to the base sequence 606 can be preprocessed and provided to the residual components of the neural network component 104, such as the first residual component 614 up to the Nth residual component 616. The residual components of the neural network component 104 can determine a probability of an amino acid being located at a given position of the base sequence 606 and determine a loss value 608 for the given position based on the probability. In various examples, the probability can be modified as the data generated by a previous residual component is fed into a subsequent residual component until the Nth residual component 616 provides an output indicating a final probability used by the neural network component 104 to generate a loss value 608.

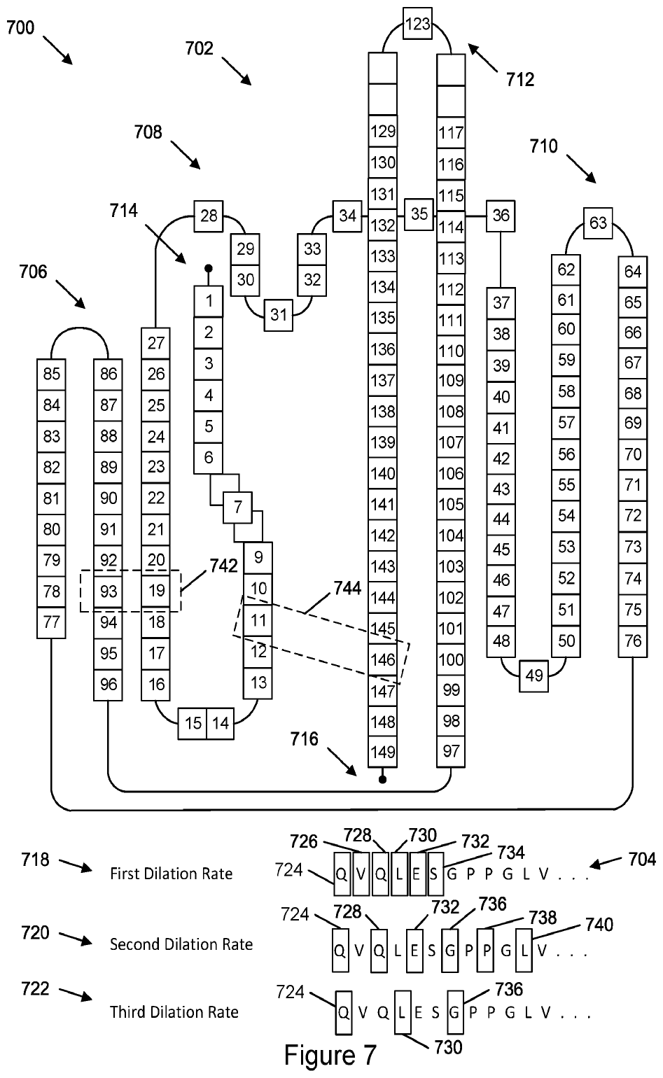

FIG. 7 illustrates an example framework 700 that includes a schema 702 for arranging amino acids of antibodies and the use of different dilation rates to traverse an example amino acid sequence 704 arranged according to the schema 702, in accordance with one or more implementations. In one or more examples, the schema 702 can indicate a number of positions of amino acids of an antibody and an arrangement of the positions such that physical features of the antibody can be reflected in the schema 702. For example, the schema 702 can indicate an outer loop region 706 of an antibody and a first complementarity determining region 708 of an antibody. The schema 702 can also indicate a second complementarity determining region 710 and a third complementarity determining region 712. In addition, the schema 702 can indicate an N-terminus 714 and a C-terminus 716. In various examples, the schema 702 can include a given number of positions, such as 149, for example. In one or more scenarios, an antibody can have fewer amino acids than the given number of positions of the schema 702. In these situations, one or more positions of the schema 702 can be blank and not occupied by an amino acid.

An antibody sequence, such as the illustrative antibody sequence 704, can be analyzed and traversed according to the schema 702 by a convolutional neural network according to a number of dilation rates. In the illustrative example of FIG. 7, the antibody sequence 704 can be analyzed and traversed according to a first dilation rate 718, a second dilation rate 720, and a third dilation rate 722 using a kernel that includes three amino acids. To illustrate, in situations where the antibody sequence 704 is analyzed and traversed according to the first dilation rate 718, three consecutive positions can be analyzed at a time. For example, a first position 724, a second position 726, and a third position 728 can be analyzed together followed by an analysis of a fourth position 730, a fifth position 732, and a sixth position 734. The analysis of the antibody sequence 704 according to the first dilation rate 718 can then proceed with subsequent sets of three consecutive amino acids.

Additionally, when the antibody sequence 704 is analyzed and traversed according to the second dilation rate 720, three positions can be analyzed at a given time with one position skipped between each of the positions being analyzed. For example, the first position 724, the third position 728, and the fifth position 732 can be analyzed together. Additionally, the analysis can move to a seventh position 736, an eighth position 738, and a ninth position 740 with one position skipped between the seventh position 736 and the eighth position 738 and one position skipped between the eighth position 738 and the ninth position 740. The analysis of the antibody sequence 704 according to the second dilation rate 720 can then proceed with subsequent sets of three amino acids with one amino acid skipped between the positions being analyzed.

Further, when the antibody sequence 704 is analyzed and traversed according to the third dilation rate 722, three positions can be analyzed at a given time with two positions skipped between each of the positions being analyzed. To illustrate, the first position 724, the fourth position 730, and the seventh position 736 can be analyzed together. The analysis of the antibody sequence 704 according to the third dilation rate 722 can proceed with subsequent sets of three amino acids with two amino acids skipped between the positions being analyzed.

By using different dilation rates to analyze antibody sequences arranged according to the schema, interactions between amino acids at positions that are remotely located in a one-dimensional chain, but that are proximate due to the folding of the antibody can be captured. For example, analyzing an antibody sequence arranged according to the schema 702 using multiple, different dilation rates can determine interactions between the amino acids located at position 19 and position 93 indicated by a region 742 that may not otherwise have been determined in situations where a single dilation rate is used. In an additional example, analyzing an antibody sequence arranged according to the schema 702 using multiple, different dilations rates can determine interactions between the amino acids located at position 11 and position 146 indicated by a region 744. In both examples, there are many positions located between the amino acids that are interacting, but the type of amino acid located at the given positions can have an impact on at least one of biophysical properties or structural features of the antibody due to the proximity of the amino acids in the antibody molecule itself due to the folding of the antibody. In scenarios where these interactions are not accounted for, the analysis of the antibodies with respect to at least one of one or more biophysical properties or one or more structure features can be inaccurate. Thus, the techniques described herein increase the accuracy of the analysis performed by the protein generating system 102 of FIGS. 1, 2, 3, 4, 5, and 6.

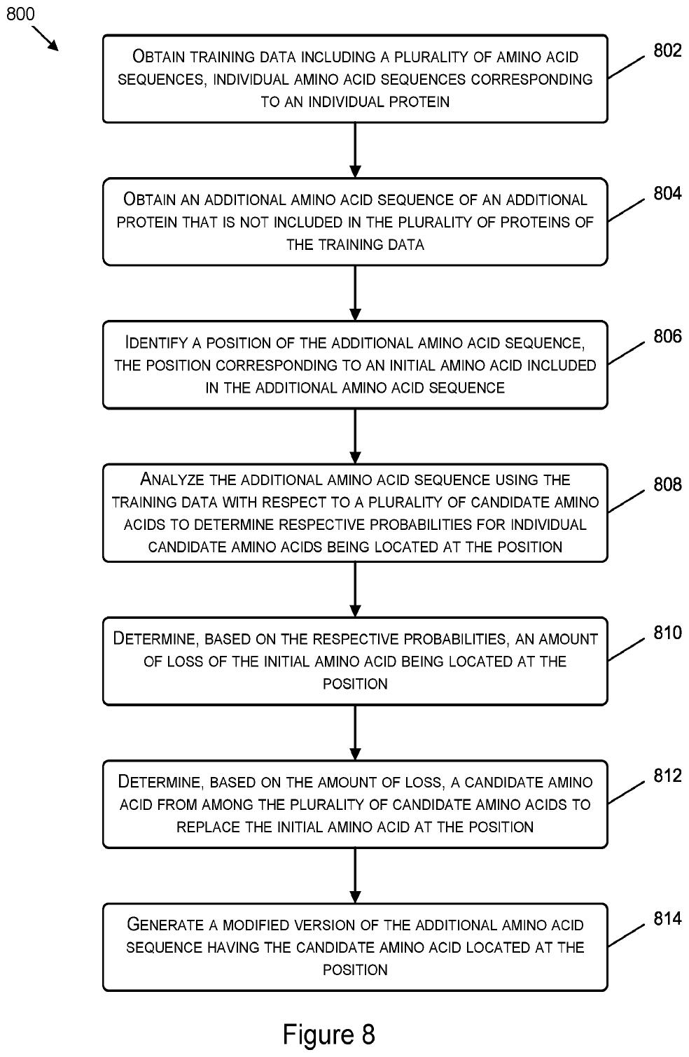

FIG. 8 illustrates an example process for generating amino acid sequences of proteins using machine learning techniques. The example process is illustrated as a collection of blocks in logical flow graphs, which represent sequences of operations that can be implemented in hardware, software, or a combination thereof. The blocks are referenced by numbers. In the context of software, the blocks represent computer-executable instructions stored on one or more computer-readable media that, when executed by one or more processing units (such as hardware microprocessors), perform the recited operations. Generally, computer-executable instructions include routines, programs, objects, components, data structures, and the like that perform particular functions or implement particular data types. The order in which the operations are described is not intended to be construed as a limitation, and any number of the described blocks can be combined in any order and/or in parallel to implement the process.

FIG. 8 is a flow diagram illustrating an example process 800 to generate protein sequences using a neural network, in accordance with one or more implementations. The process 800 can include, at 802, obtaining training data including a plurality of amino acid sequences. Individual amino acids sequences of the plurality of amino acid sequences can correspond to individual proteins. Additionally, the training data can include amino acid sequences of proteins having one or more specified biophysical properties. Further, the training data can include amino acid sequences of proteins having one or more specified structural features. That is, the training data can be generated and/or selected such that the amino acid sequences included in the training data correspond to proteins that have at least one of one or more desired biophysical properties or one or more desired structural features. In one or more illustrative examples, the training data can be generated by one or more generative adversarial networks. In various examples, the one or more generative adversarial networks can be trained using one or more transfer learning techniques such that the generative adversarial networks produce amino acid sequences of proteins having at least one of desired values of one or more biophysical properties or one or more structural features.

At 804, the process 800 can include obtaining an additional amino acid sequence of an additional protein that is not included in the plurality of proteins of the training data. The additional amino acid sequence can include abase sequence that is to be analyzed in accordance with the training data. In one or more examples, the additional amino acid sequence can have one or more characteristics that are different from characteristics of the plurality of amino acid sequences included in the training data. For example, the additional amino acid sequence can correspond to a protein that is produced in accordance with one or more genomic regions that are non-human mammal genomic regions and the plurality of amino acid sequences included in the training data can correspond to proteins produced in accordance with one or more human genomic regions. In one or more additional examples, the additional amino acid sequence can correspond to a protein that is produced in accordance with one or more first germline genomic regions and the plurality of amino acid sequences included in the training data can correspond to proteins that are produced in accordance with one or more second germline genomic regions that are different from the one or more first germline genomic regions.

The amino acid sequences included in the training data and the additional amino acid sequence can correspond to antibodies. In one or more examples, the amino acid sequences included in the training data and the additional amino acid sequence can correspond to antibody fragments. For example, the amino acid sequences included in the training data and the additional amino acid sequence can correspond to heavy chain variable regions. In one or more additional examples, the amino acid sequences of the training data and the additional amino acid sequence can correspond to light chain variable regions. In one or more illustrative examples, the amino acid sequences of the training data and the additional amino acid sequence can correspond to kappa light chain variable regions. In one or more further illustrative examples, the amino acid sequences of the training data and the additional amino acid sequence can correspond to lambda light chain variable regions. In still other examples, the amino acid sequences of the training data and the additional amino acid sequence can correspond to afibronectin type III (FNIII) proteins, avimers, VHH domains, kinases, zinc fingers, T-cell receptors, one or more combinations thereof, and the like.

In addition, at 806, the process 800 can include identifying a position of the additional amino acid sequence where the position includes an initial amino acid included in the additional amino acid sequence. Further, the process 800 can include, at operation 808, analyzing the additional amino acid sequence using the training data with respect to a plurality of candidate amino acids to determine respective probabilities for individual candidate amino acids being located at the position. In one or more examples, a plurality of individual positions of the additional amino acid sequence can be identified and subsequently analyzed. In one or more further examples, each individual position of the additional amino acid sequence can be identified and analyzed. In various examples, the additional amino acid sequence can be arranged according to a schema and one or more positions of the additional amino acid sequence can be identified and analyzed. In scenarios where a convolutional neural network is used to analyze the additional amino acid sequence, a kernel of the convolutional neural network and a dilation rate of the convolutional neural network can be used to identify the one or more amino acids analyzed by the convolutional neural network at a given time.

In various examples, the individual position of the amino acid sequence can be analyzed with respect to 20 standard amino acids that are encoded directly by genomic regions of one or more reference human genomes. In these scenarios, twenty probabilities can be determined with individual probabilities corresponding to the individual standard amino acids. In one or more additional examples, the individual position of the amino acid sequence can be analyzed with respect to 22 proteinogenic amino acids. Additionally, in these situations, twenty-two probabilities can be determined with individual probabilities corresponding to the individual proteinogenic amino acids. In one or more additional examples, different sets of amino acids can comprise the plurality of candidate amino acids. To illustrate, the plurality of candidate amino acids can include a subset of the standard amino acids or a subset of the proteinogenic amino acids. Further, the plurality of candidate amino acids can include amino acids in addition to the standard amino acids or the proteinogenic amino acids, such as one or more nonstandard amino acids or one or more non-proteinogenic amino acids.

The process 800 can also include, at 810, determining, based on the respective probabilities, an amount of loss of the initial amino acid being located at the position. For example, the amount of loss can be determined by analyzing a probability of the initial amino acid being located at the position with respect to one or more additional probabilities of one or more additional amino acids being located at the position. The amount of loss can be a greater value in situations where the probability of the amino acid being located at the position decreases and as the probability of additional amino acids being located at the position increases. Additionally, the amount of loss can be a lower value in scenarios where the probability of the amino acid being located at the position increases and as the probability of additional amino acids being located at the position decreases.

At 812, the process 800 can include determining, based on the amount of loss, a candidate amino acid from among the plurality of candidate amino acids to replace the initial amino acid at the position. In one or more examples, a candidate amino acid can be determined to replace the initial amino acid when the loss for the initial amino acid is greater than a threshold loss value. Additionally, the probabilities of the plurality of candidate amino acids can be analyzed to determine the candidate amino acid having the greatest probability of being located at the position that can be used to replace the initial amino acid. In one or more additional examples, the candidate amino acid determined to replace the initial amino acid can minimize an overall loss value of the additional amino acid sequence.

Further, the process 800 can include, at 814, generating a modified version of the additional amino acid sequence having the candidate amino acid located at the position. The modified version of the additional amino acid sequence can be a variant of the additional amino acid base sequence. In one or more examples, the modified version of the additional amino acid sequence can include a plurality of substitutions where initial amino acids located at respective positions are replaced by candidate amino acids. In various examples, each position of the additional amino acid sequence can be analyzed to determine whether an initial amino acid located at a given position is to be replaced by a candidate amino acid. In one or more illustrative examples, the initial amino acids can be replaced by candidate amino acids to modify at least one of one or more biophysical properties or one or more structural features of the additional amino acid sequence. For example, initial amino acids located at one or more positions of the additional amino acid sequence can be replaced to modify one or more solubility characteristics of the additional amino acid sequence or to modify one or more thermostability characteristics of the additional amino acid sequence, such as a melting point of the additional amino acid sequence. In one or more additional examples, initial amino acids located at one or more positions of the additional amino acid sequence can be replaced to modify a number of amino acids included in one or more negatively charged regions of the additional amino acid sequence or to modify a number of amino acids included in one or more hydrophobic regions of the additional amino acid sequence. In one or more further examples, the initial amino acids located at one or more positions of the additional amino acid sequence can be replaced to humanize the additional amino acid sequence. The initial amino acids located at one or more positions of the additional amino acid sequence can also be replaced to cause a protein that corresponds to the additional amino acid sequence and that is produced in accordance with one or more first germline genomic regions to have characteristics that are more like proteins produced in accordance with one or more second germline genomic regions.

In one or more examples, an additional amount of loss can be determined for the modified version of the additional amino acid sequence. The amount of loss for the additional amino acid sequence and the additional amount of loss of the modified version of the additional amino acid sequence can be used to evaluate the additional amino acid sequence and the modified version of the additional amino acid sequence with respect to at least one of one or more biophysical properties or one or more structural features. For examples, the amount of loss can indicate an amount of difference between amino acid sequences included in the training data with respect to the additional amino acid sequence and the modified version of the additional amino acid sequence. In various examples, the amount of loss can indicate how closely one or more biophysical properties of proteins that correspond to additional amino acid sequence and the modified version of the additional amino acid sequence correspond to the plurality of proteins included in the training data. In one or more scenarios, the amount of loss can be used to determine scores for amino acid sequences that can be used to evaluate the amino acid sequences with respect to at least one of one or more biophysical properties, one or more structural features, or one or more germline sequences of proteins that correspond to the amino acid sequences. In at least some examples, the amino acid sequences can be used to synthesize or otherwise express proteins that correspond to the amino acid sequences. In these scenarios, the biophysical properties of the expressed proteins can be evaluated with respect to the predicted values of the biophysical properties.

The amount of loss can be different with respect to different biophysical properties, structural features, or germline sequences. For example, the additional amino acid sequence and/or the modified version of the additional amino acid sequence can have first scores in relation to a first biophysical property or a first structural feature and second scores in relation to a second biophysical property or a second structural feature. To illustrate, first training data can include first amino acid sequences of first proteins having a first set of characteristics and second training data can include second amino acid sequences of second proteins having a second set of characteristics. In one or more illustrative examples, the first set of characteristics can correspond to at least one of one or more first values of one or more biophysical properties, one or more first structural features, or one or more first germline sequences. and the second set of characteristics can correspond to at least one of one or more second values of one or more biophysical properties, one or more second structural features, or one or more second germline sequences. As a result of changes to the characteristics of the training data, the amount of loss and scoring for amino acid sequences can be different because the probabilities of amino acids being located in respective positions of the amino acids sequences can be different in relation to the different training datasets.

In one or more illustrative examples, the amount of loss determined with respect to amino acid sequences can be used to generate a score that indicates to an amount of unfolding of proteins that correspond to the amino acid sequences. In various examples, an amount of unfolding of proteins can be determined using differential scanning fluorimetry. In one or more examples, an amount of chemical unfolding of proteins can be determined based on a measure of unfolding induced by guanidine hydrochloride (GdnHCl). In one or more additional illustrative examples, the amount of loss determined with respect to amino acid sequences can be used to generate a score that indicates a percentage of high molecular weight moieties of proteins that correspond to the amino acid sequences. In at least some examples, scores of amino acid sequences with respect to one or more biophysical properties, one or more structural properties, or correspondence with one or more germline amino acid sequences can be used to determine a measure of stability of proteins that correspond to the amino acid sequences. In one or more further examples, scores of amino acid sequences with respect to one or more biophysical properties, one or more structural properties, or correspondence with one or more germline amino acid sequences can be used to determine an amount of binding of proteins having the amino acid sequences to target molecules. In one or more examples, the scores generated for amino acid sequences with respect to one or more biophysical properties, one or more structural properties, or correspondence with one or more germline amino acid sequences can be used to determine whether or not to synthesize and/or otherwise express proteins that correspond to the amino acid sequence for a given purpose or function.

In still other examples, scores or other quantitative measures generated based on the amount of loss for amino acid sequences can be used to optimize amino acid sequences and/or characteristics of proteins by evaluating the scores in conjunction with various criteria that correspond to biophysical properties and/or structural features of proteins. For example, proteins having hydrophobic patches of a given size, such as greater than 150 Angstroms² can be assigned a first score and proteins having hydrophobic patches of an additional size, such as greater than 250 Angstroms² can be assigned a second score. In these situations, the score with regard to size of hydrophobic patches can be analyzed in conjunction with a quantitative measure determined based on an amount of loss determined for the amino acid sequence of the protein to determine a measure of stability of the protein and/or a likelihood of the protein binding to a target molecule. In one or more additional examples, other biophysical properties and/or structural features can be analyzed in conjunction with the quantitative measures determined based on an amount of loss for an amino acid sequence, such as isoelectric point measurements, differential scanning fluorimetry measurements, self-interaction nanoparticle spectroscopy measurements, combinations thereof, and the like. Amino acid sequences can be ranked based on their respective scores in relation to one or more criteria, such as solubility in one or more solutions, likelihood of binding a target molecule, or likelihood of unfolding in a given environment.

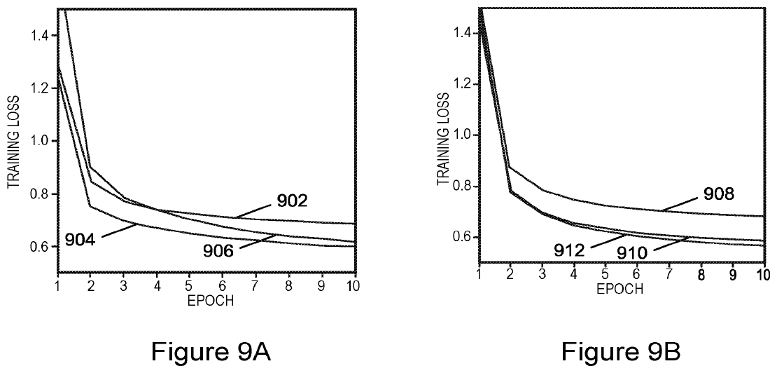

FIG. 9A illustrates training loss that takes place in response to training a neural network using existing techniques and FIG. 9B illustrates training loss that takes place in response to training a neural network in accordance with techniques described herein. For example, FIG. 9A shows an amount of training loss after a number of epochs for a convolutional neural network configured according to existing techniques having a single layer 902, 11 layers 904, and 21 layers 906. The training loss for a convolutional neural network having 21 layers 906 after 4 or more epochs is greater than the training loss for the convolutional neural network having 11 layers 904. Thus, FIG. 9A indicates that attempting to implement convolutional neural networks according to existing techniques to modify amino acid sequences as described herein result in inefficiencies in the training of the convolutional neural networks that can result in inaccurate results being produced from the convolutional neural network implemented according to existing techniques. However, FIG. 9B shows that the training loss for a convolutional neural network implemented according to techniques described herein has a training loss for a single layer 908, 11 layers 910, and 21 layers 912 that is different from that of convolutional neural networks implemented according to existing techniques. To illustrate, the training loss of a convolutional neural network having 21 layers 912 implemented according to techniques described herein is less than the training loss of the convolutional neural network having 11 layers 910. Accordingly, convolutional neural networks implemented according to techniques described herein are more efficient and more accurate than convolutional neural networks that are implemented according to existing techniques for the purposes of determining loss values of positions of base amino acid sequences and determining changes to the base amino acid sequences to generate variant amino acid sequences.

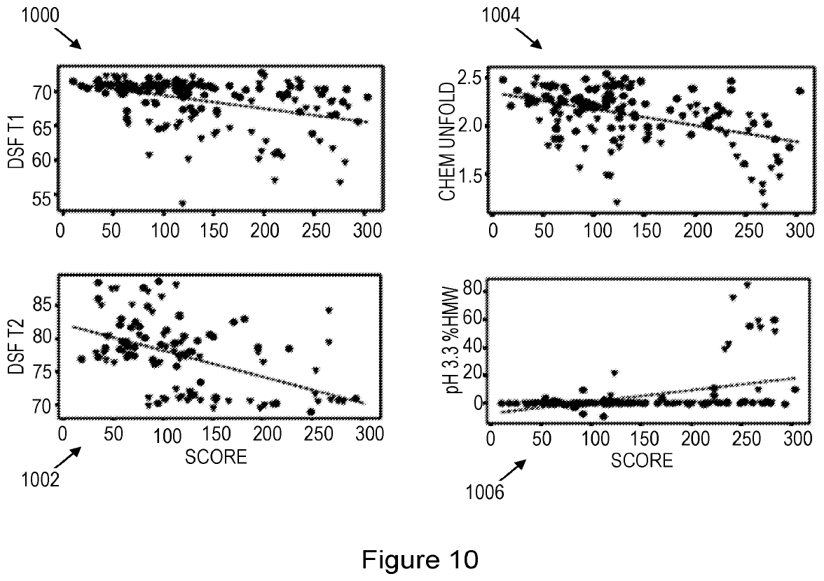

FIG. 10 includes a number of scatter plots indicating correlations between loss scores generated according to implementations described herein and experimental measurements for a set of antibodies. In each of the scatter plots, the circles represent antibodies having data derived from at least one of a clinical setting or from the Protein Data Bank and the triangles represent variants of a subset of the antibodies represented by the circles. To determine the aggregate loss scores, neural network models were trained according to implementations described herein for heavy chain antibody sequences, kappa light chain antibody sequences, and lambda light chain antibody sequences to predict amino acid probabilities at the positions of the antibody sequences. The models were applied to each antibody sequence in the experimental test set, and summed across sequence the residue-wise loss scores derived from the model predictions. The models were trained on millions of human antibody sequences deposited in the observed antibody space (OAS) database.

FIG. 10 includes a first scatter plot 1000 indicating first thermal stability measures for a number of antibodies with respect to loss scores of variant sequences where the loss scores and variant sequences are generated according to implementations described herein. The measures of thermal stability are generated using differential scanning fluorimetry. FIG. 10 also includes a second scatter plot 1002 indicating second thermal stability measures for a number of antibodies with respect to loss scores for variant sequences where the loss scores and variant sequences are generated according to implementations described herein. The measures of thermal stability are generated using differential scanning fluorimetry. Additionally, FIG. 10 includes a third scatter plot 1004 indicating measures of chemical unfolding for a number of antibodies with respect to loss scores of variant sequences where the loss scores and variant sequences are generated according to implementations described herein. Further, FIG. 10 includes a fourth scatter plot 1006 indicating measures of percentage of high molecular weight segments at a pH of 3.3 for a number of antibodies with respect to loss scores of variant sequences where the loss scores and variant sequences are generated according to implementations described herein.

The scatter plots 1000, 1002, 1004, 1006 indicate that the loss scores for the variants correlate with experimental measurements of thermal, chemical, and pH stability for a relatively large number of antibodies. Additionally, the scatter plots 1000, 1002, 1004, 1006 indicate that the models used to generate the loss scores encode information about antibody stability without being trained on stability related data, but with training on antibody sequence data.

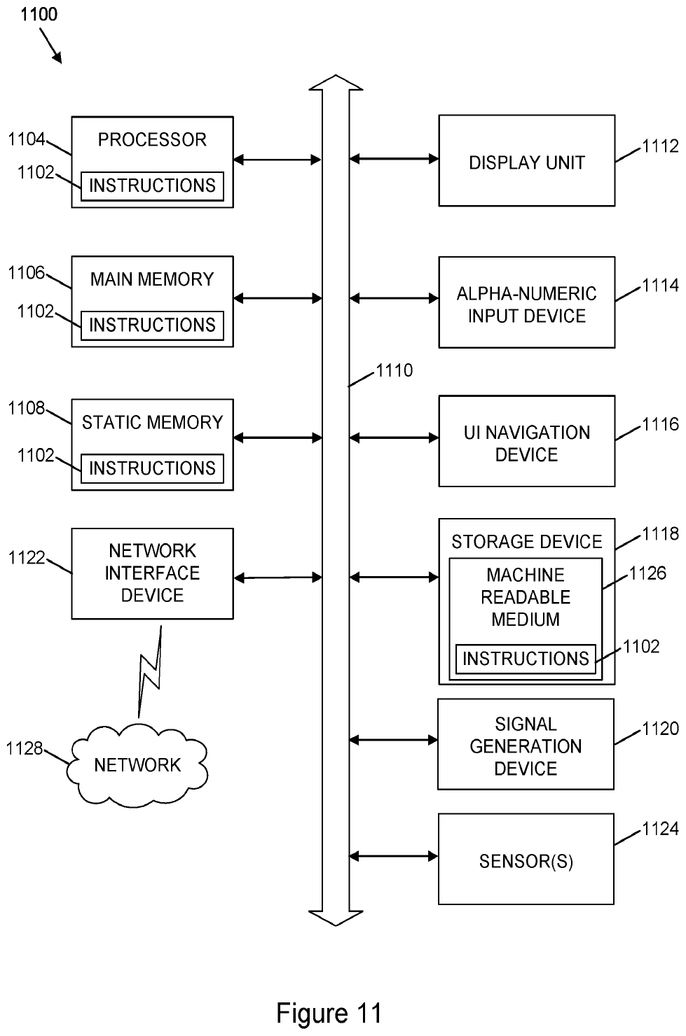

FIG. 11 illustrates a diagrammatic representation of a computing device 1100 in the form of a computer system within which a set of instructions may be executed for causing the computing device 1100 to perform any one or more of the methodologies discussed herein, according to an example, according to an example implementation. Specifically, FIG. 11 shows a diagrammatic representation of the computing device 1100 in the example form of a computer system, within which instructions 1102 (e.g., software, a program, an application, an applet, an app, or other executable code) for causing the computing device 1100 to perform any one or more of the methodologies discussed herein may be executed. For example, the instructions 1102 may cause the computing device 1100 to implement the frameworks 100, 200, 300, 400, 500, 600, 700 described with respect to FIGS. 1, 2, 3, 4, 5, 6, and 7, respectively, and to execute the methods 800, 900 described with respect to FIGS. 8 and 9, respectively.

The instructions 1102 transform the general, non-programmed computing device 1100 into a particular computing device 1100 programmed to carry out the described and illustrated functions in the manner described. In alternative implementations, the computing device 1100 operates as a standalone device or may be coupled (e.g., networked) to other machines. In a networked deployment, the computing device 1100 may operate in the capacity of a server machine or a client machine in a server-client network environment, or as a peer machine in a peer-to-peer (or distributed) network environment. The computing device 1100 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1102, sequentially or otherwise, that specify actions to be taken by the computing device 1100. Further, while only a single computing device 1100 is illustrated, the term “machine” shall also be taken to include a collection of computing devices 1100 that individually or jointly execute the instructions 1102 to perform any one or more of the methodologies discussed herein.

Examples of computing device 1100 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In implementations in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service”

(SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example implementations (e.g., apparatus, systems, or methods) can be implemented in digital electronic circuitry, in computer hardware, in firmware, in software, or in any combination thereof. Example implementations can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In implementations deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., computing device 1100) and software architectures that can be deployed in example implementations.

In an example, the computing device 1100 can operate as a standalone device or the computing device 1100 can be connected (e.g., networked) to other machines.

In a networked deployment, the computing device 1100 can operate in the capacity of either a server or a client machine in server-client network environments. In an example, computing device 1100 can act as a peer machine in peer-to-peer (or other distributed) network environments. The computing device 1100 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the computing device 1100. Further, while only a single computing device 1100 is illustrated, the term “computing device” shall also be taken to include any collection of machines that individually or jointly execute a set (or multiple sets) of instructions to perform any one or more of the methodologies discussed herein.

Example computing device 1100 can include a processor 1104 (e.g., a central processing unit CPU), a graphics processing unit (GPU) or both), a main memory 1106 and a static memory 1108, some or all of which can communicate with each other via a bus 1110. The computing device 1100 can further include a display unit 1112, an alphanumeric input device 1114 (e.g., a keyboard), and a user interface (UI) navigation device 1116 (e.g., a mouse). In an example, the display unit 1112, input device 1114 and UI navigation device 1116 can be a touch screen display. The computing device 1100 can additionally include a storage device (e.g., drive unit) 1118, a signal generation device 1120 (e.g., a speaker), a network interface device 1122, and one or more sensors 1124, such as a global positioning system (GPS) sensor, compass, accelerometer, or another sensor.

The storage device 1118 can include a machine readable medium 1126 on which is stored one or more sets of data structures or instructions 1102 (e.g., software) embodying or utilized by any one or more of the methodologies or functions described herein. The instructions 1102 can also reside, completely or at least partially, within the main memory 1106, within static memory 1108, or within the processor 1104 during execution thereof by the computing device 1100. In an example, one or any combination of the processor 1104, the main memory 1106, the static memory 1108, or the storage device 1118 can constitute machine readable media.

While the machine readable medium 1126 is illustrated as a single medium, the term “machine readable medium” can include a single medium or multiple media (e.g., a centralized or distributed database, and/or associated caches and servers) that configured to store the one or more instructions 1102. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine-readable media can include non-volatile memory, including, by way of example, semiconductor memory devices (e.g., Electrically Programmable Read-Only Memory

(EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 1102 can further be transmitted or received over a communications network 1128 using a transmission medium via the network interface device 1122 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

As used herein, a component, such as the neural network component 106 and the variant generating component 108 can refer to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A numbered non-limiting list of aspects of the present subject matter is presented below.

Aspect 1. A method comprising: obtaining, by a computing system including one or more computing devices having one or more processors and memory, training data including a plurality of amino acid sequences, individual amino acid sequences of the plurality of amino acid sequences corresponding to an individual protein of a plurality of proteins; obtaining, by the computing system, an additional amino acid sequence of an additional protein that is not included in the plurality of proteins; identifying, by the computing system, a position of the additional amino sequence, the position corresponding to an initial amino acid included in the additional amino acid sequence; analyzing the additional amino acid sequence, by the computing system and using the training data, with respect to a plurality of candidate amino acids to determine respective probabilities for individual candidate amino acids of the plurality of candidate amino acids being located at the position; determining, by the computing system and based on the respective probabilities, an amount of loss of the initial amino acid being located at the position; determining, by the computing system and based on the amount of loss, a candidate amino acid from among the plurality of candidate amino acids to replace the initial amino acid at the position; and generating, by the computing system, a modified version of the additional amino acid sequence having the candidate amino acid located at the position.

Aspect 2. The method of aspect 1, wherein a value of a biophysical property of a variant protein corresponding to the modified version of the amino acid sequence is greater than an additional value of the biophysical property of the additional protein corresponding to the initial amino acid sequence.

Aspect 3. The method of claim 1 or 2, wherein a measure of stability of a variant protein corresponding to the modified version of the amino acid sequence is greater than an additional measure of stability of the additional protein corresponding to the initial amino acid sequence.

Aspect 4. The method of any one of claims 1-3, comprising: determining, by the computing system, that the amount of loss of the initial amino acid being located at the position is at least a threshold amount of loss; and determining, by the computing system, that the initial amino acid is to be replaced based on the amount of loss of the initial amino acid being at least the threshold amount of loss.

Aspect 5. The method of any one of claims 1-3, comprising: determining, by the computing system, that a first probability of the candidate amino acid being located at the position is greater than a second probability of the initial amino acid being located at the position; and determining, by the computing system, that the initial amino acid is to be replaced based on the first probability being greater than the second probability.

Aspect 6. The method of any one of claims 1-5, comprising: performing, by the computing system, a first training process using first additional training to produce a trained generating component of a generative adversarial network, the first additional training data including a first additional plurality of amino acid sequences of first additional proteins; obtaining, by the computing system, a second additional training data that includes a second additional plurality of amino acid sequences of second additional proteins, the second additional proteins including a greater number of proteins having at least one of a structural feature or a biophysical property than the first additional plurality of first proteins included in the first additional training data; performing, by the computing system and using the second additional training data, a second training process for a generative adversarial network that includes the trained generating component; and producing, by the computing system, an additional trained generating component in relation to the second training process, the additional trained generating component generating at least a portion of the training data.

Aspect 7. The method of any of claims 1-6, wherein a convolutional neural network is implemented to analyze the additional amino acid sequence and determine the respective probabilities, the convolutional neural network having a plurality of residual layers.

Aspect 8. The method of claim 7, wherein: the convolutional neural network includes a first residual component that provides at least a portion of the output of the first residual component to a second residual component; the first residual component analyzes amino acid sequences in accordance with a first dilation rate; and the second residual component analyzes amino acid sequences in accordance with a second dilation rate.

Aspect 9. The method of any one of claims 1-8, comprising: determining, by the computing system, individual amounts of loss for a plurality of positions of the additional amino acid sequence; determining, by the computing system, an aggregate amount of loss based on the individual amounts of loss; and determining, by the computing system, a score for the additional amino acid sequence based on the aggregate amount of loss.

Aspect 10. The method of claim 9, comprising: determining, by the computing system, additional individual amounts of loss for an additional plurality of positions of the modified version of additional amino acid sequence; determining, by the computing system, an additional aggregate amount of loss based on the additional individual amounts of loss; and determining, by the computing system, an additional score for the modified version of the additional amino acid sequence.

Aspect 11. The method of claim 10, wherein: the score for the additional amino acid sequence is lower than the additional score for the modified version of the additional amino acid sequence; and a first value of a biophysical property for the additional amino acid sequence is less than a second value of the biophysical property for the modified version of the additional amino acid sequence.

Aspect 12. The method of claim 10, wherein: the score for the additional amino acid sequence is lower than the additional score for the modified version of the additional amino acid sequence; and the modified version of the additional amino acid sequence has a greater amount of homology with respect to amino acid sequences of proteins generated in accordance with one or more germline genomic regions than the additional amino acid sequence.

Aspect 13. A system comprising: one or more hardware processing units; and one or more non-transitory memory devices storing computer-readable instructions that, when executed by the one or more hardware processing units, cause the system to perform operations comprising: obtaining training data including a plurality of amino acid sequences, individual amino acid sequences of the plurality of amino acid sequences corresponding to an individual protein of a plurality of proteins; obtaining an additional amino acid sequence of an additional protein that is not included in the plurality of proteins; identifying a position of the additional amino sequence, the position corresponding to an initial amino acid included in the additional amino acid sequence; analyzing the additional amino acid sequence, using the training data, with respect to a plurality of candidate amino acids to determine respective probabilities for individual candidate amino acids of the plurality of candidate amino acids being located at the position; determining, based on the respective probabilities, an amount of loss of the initial amino acid being located at the position; determining, based on the amount of loss, a candidate amino acid from among the plurality of candidate amino acids to replace the initial amino acid at the position; and generating a modified version of the additional amino acid sequence having the candidate amino acid located at the position.

Aspect 14. The system of aspect 13, wherein a value of a biophysical property of a variant protein corresponding to the modified version of the amino acid sequence is greater than an additional value of the biophysical property of the additional protein corresponding to the initial amino acid sequence.

Aspect 15. The system of aspect 13 or 14, wherein a measure of stability of a variant protein corresponding to the modified version of the amino acid sequence is greater than an additional measure of stability of the additional protein corresponding to the initial amino acid sequence.

Aspect 16. The system of any one of aspects 13-15, wherein the one or more non-transitory memory devices store additional computer-readable instructions that, when executed by the one or more hardware processing units, cause the system to perform additional operations comprising: determining that the amount of loss of the initial amino acid being located at the position is at least a threshold amount of loss; and determining that the initial amino acid is to be replaced based on the amount of loss of the initial amino acid being at least the threshold amount of loss.

Aspect 17. The system of any one of aspects 13-15, wherein the one or more non-transitory memory devices store additional computer-readable instructions that, when executed by the one or more hardware processing units, cause the system to perform additional operations comprising: determining that a first probability of the candidate amino acid being located at the position is greater than a second probability of the initial amino acid being located at the position; and determining that the initial amino acid is to be replaced based on the first probability being greater than the second probability.

Aspect 18. The system of any one of aspects 13-17, wherein the one or more non-transitory memory devices store additional computer-readable instructions that, when executed by the one or more hardware processing units, cause the system to perform additional operations comprising: performing a first training process using first additional training to produce a trained generating component of a generative adversarial network, the first additional training data including a first additional plurality of amino acid sequences of first additional proteins; obtaining a second additional training data that includes a second additional plurality of amino acid sequences of second additional proteins, the second additional proteins including a greater number of proteins having at least one of a structural feature or a biophysical property than the first additional plurality of first proteins included in the first additional training data; performing, using the second additional training data, a second training process for a generative adversarial network that includes the trained generating component; and producing an additional trained generating component in relation to the second training process, the additional trained generating component generating at least a portion of the training data.

Aspect 19. The system of any of aspects 13-18, wherein a convolutional neural network is implemented to analyze the additional amino acid sequence and determine the respective probabilities, the convolutional neural network having a plurality of residual layers.

Aspect 20. The system of aspect 19, wherein: the convolutional neural network includes a first residual component that provides at least a portion of the output of the first residual component to a second residual component; the first residual component analyzes amino acid sequences in accordance with a first dilation rate; and the second residual component analyzes amino acid sequences in accordance with a second dilation rate.

Aspect 21. The system of any one of aspects 13-20, wherein the one or more non-transitory memory devices store additional computer-readable instructions that, when executed by the one or more hardware processing units, cause the system to perform additional operations comprising: determining individual amounts of loss for a plurality of positions of the additional amino acid sequence; determining an aggregate amount of loss based on the individual amounts of loss; and determining a score for the additional amino acid sequence based on the aggregate amount of loss.

Aspect 22. The system of aspect 21, wherein the one or more non-transitory memory devices store additional computer-readable instructions that, when executed by the one or more hardware processing units, cause the system to perform additional operations comprising: determining additional individual amounts of loss for an additional plurality of positions of the modified version of additional amino acid sequence; determining an additional aggregate amount of loss based on the additional individual amounts of loss; and determining an additional score for the modified version of the additional amino acid sequence.

Aspect 23. The system of aspect 22, wherein: the score for the additional amino acid sequence is lower than the additional score for the modified version of the additional amino acid sequence; and a first value of a biophysical property for the additional amino acid sequence is less than a second value of the biophysical property for the modified version of the additional amino acid sequence.

Aspect 24. The system of aspect 22, wherein: the score for the additional amino acid sequence is lower than the additional score for the modified version of the additional amino acid sequence; and the modified version of the additional amino acid sequence has a greater amount of homology with respect to amino acid sequences of proteins generated in accordance with one or more germline genomic regions than the additional amino acid sequence.

Aspect 25. A system comprising: one or more hardware processing units; and one or more non-transitory memory devices storing computer-readable instructions that, when executed by the one or more hardware processing units, cause the system to perform operations comprising: obtaining training data including a plurality of amino acid sequences, individual amino acid sequences of the plurality of amino acid sequences corresponding to an antibody fragment of a plurality of antibody fragments; obtaining an additional amino acid sequence of an additional antibody component that is not included in the plurality of antibody components; identifying a position of the additional amino sequence, the position corresponding to an initial amino acid included in the additional amino acid sequence; analyzing the additional amino acid sequence, using the training data, with respect to a plurality of candidate amino acids to determine respective probabilities for individual candidate amino acids of the plurality of candidate amino acids being located at the position; and determining, based on the respective probabilities, an amount of loss of the initial amino acid being located at the position.

Aspect 26. The system of aspect 25, wherein a convolutional neural network is implemented to analyze the additional amino acid sequence to determine respective probabilities for the individual candidate amino acid sequences and to determine the amount of loss of the initial amino acid being located at the position.

Aspect 27. The system of aspect 26, wherein the convolutional neural network includes a plurality of residual layers and a plurality of one-dimensional convolutional layers.

Aspect 28. The system of aspect 26, wherein the convolutional neural network implements one or more first models that analyze heavy chain variable regions of antibody sequences and one or more second models that analyze light chain variable regions of antibody sequences.

Aspect 29. The system of aspect 28, wherein the one or more second models include at least one second model to analyze kappa light chain variable regions of antibody sequences and at least one additional second model to analyze lambda light chain variable regions of antibody sequences.

Aspect 30. The system of aspect 28 or 29, wherein the one or more non-transitory memory devices store additional computer-readable instructions that, when executed by the one or more hardware processing units, cause the system to perform additional operations comprising: generating, based on a first base sequence, a first amino acid sequence corresponding to a first variant sequence of a heavy chain variable region; generating, based on a second base sequence, a second amino acid sequence corresponding to a second variant sequence of a light chain variable region; and combining the first amino acid sequence and the second amino acid sequence to generate an antibody amino acid sequence that includes the heavy chain variable region and the light chain variable region.

Aspect 31. A method comprising: obtaining, by a computing system including one or more computing devices having one or more processors and memory, training data including a plurality of amino acid sequences, individual amino acid sequences of the plurality of amino acid sequences corresponding to an antibody fragment of a plurality of antibody fragments; obtaining, by the computing system, an additional amino acid sequence of an additional antibody component that is not included in the plurality of antibody components; identifying, by the computing system, a position of the additional amino sequence, the position corresponding to an initial amino acid included in the additional amino acid sequence; analyzing, by the computing system, the additional amino acid sequence, using the training data, with respect to a plurality of candidate amino acids to determine respective probabilities for individual candidate amino acids of the plurality of candidate amino acids being located at the position; and determining, by the computing system and based on the respective probabilities, an amount of loss of the initial amino acid being located at the position.

Aspect 32. The method of aspect 31, wherein a convolutional neural network is implemented to analyze the additional amino acid sequence to determine respective probabilities for the individual candidate amino acid sequences and to determine the amount of loss of the initial amino acid being located at the position.

Aspect 33. The method of aspect 32, wherein the convolutional neural network includes a plurality of residual layers and a plurality of one-dimensional convolutional layers.

Aspect 34. The method of aspect 32, wherein the convolutional neural network implements one or more first models that analyze heavy chain variable regions of antibody sequences and one or more second models that analyze light chain variable regions of antibody sequences.

Aspect 35. The method of aspect 34, wherein the one or more second models include at least one second model to analyze kappa light chain variable regions of antibody sequences and at least one additional second model to analyze lambda light chain variable regions of antibody sequences.

Aspect 36. The method of aspect 34 or 35, comprising: generating, by the computing system and based on a first base sequence, a first amino acid sequence corresponding to a first variant sequence of a heavy chain variable region; generating, by the computing system and based on a second base sequence, a second amino acid sequence corresponding to a second variant sequence of a light chain variable region; and combining, by the computing system, the first amino acid sequence and the second amino acid sequence to generate an antibody amino acid sequence that includes the heavy chain variable region and the light chain variable region.

Aspect 37. A method comprising: obtaining, by a computing system including one or more computing devices having one or more processors and memory, training data including a plurality of amino acid sequences, individual amino acid sequences of the plurality of amino acid sequences corresponding to an individual protein of a plurality of proteins; obtaining, by the computing system, a base amino acid sequence of at least a portion of a first additional protein that is not included in the plurality of proteins; obtaining, by the computing system, a grafting sequence of at least a portion of a second additional protein that is not included in the plurality of proteins; determining, by the computing system and based on position modification data, a plurality of first positions of the base sequence that include first amino acids that are to be modified; determining, by the computing system and based on the position modification data, a plurality of second positions of the grafting sequence that include second amino acids that are to replace the first amino acids; generating, by the computing system, a combined amino acid sequence that includes a modified version of the base sequence with the first amino acids being replaced by the second amino acids; analyzing the combined amino acid sequence, by the computing system and using the training data, with respect to a plurality of candidate amino acids to determine respective probabilities for individual candidate amino acids of the plurality of candidate amino acids being located at a position of the combined sequence; determining, by the computing system and based on the respective probabilities, an amount of loss of an initial amino acid being located at the position. determining, by the computing system and based on the amount of loss, a candidate amino acid from among the plurality of candidate amino acids to replace the initial amino acid at the position; and generating, by the computing system, a modified version of the additional amino acid sequence having the candidate amino acid located at the position.

Aspect 38. The method of aspect 37, wherein the first additional protein is produced according to one or more human genomic regions; and the second additional protein is produced according to one or more non-human mammalian genomic regions.

Aspect 39. A system comprising: one or more hardware processing units; and one or more non-transitory memory devices storing computer-readable instructions that, when executed by the one or more hardware processing units, cause the system to perform operations comprising: obtaining training data including a plurality of amino acid sequences, individual amino acid sequences of the plurality of amino acid sequences corresponding to an individual protein of a plurality of proteins; obtaining a base amino acid sequence of at least a portion of a first additional protein that is not included in the plurality of proteins; obtaining a grafting sequence of at least a portion of a second additional protein that is not included in the plurality of proteins; determining, based on position modification data, a plurality of first positions of the base sequence that include first amino acids that are to be modified; determining, based on the position modification data, a plurality of second positions of the grafting sequence that include second amino acids that are to replace the first amino acids; generating, by the computing system, a combined amino acid sequence that includes a modified version of the base sequence with the first amino acids being replaced by the second amino acids; analyzing the combined amino acid sequence, using the training data, with respect to a plurality of candidate amino acids to determine respective probabilities for individual candidate amino acids of the plurality of candidate amino acids being located at a position of the combined sequence; determining, based on the respective probabilities, an amount of loss of an initial amino acid being located at the position. determining, based on the amount of loss, a candidate amino acid from among the plurality of candidate amino acids to replace the initial amino acid at the position; and generating a modified version of the additional amino acid sequence having the candidate amino acid located at the position.

Aspect 40. The system of aspect 39, wherein the first additional protein is produced according to one or more human genomic regions; and the second additional protein is produced according to one or more non-human mammalian genomic regions.

What is claimed is:

1. A method comprising:
obtaining, by a computing system including one or more computing devices having one or more processors and memory, training data including a plurality of amino acid sequences, individual amino acid sequences of the plurality of amino acid sequences corresponding to an individual protein of a plurality of proteins;
obtaining, by the computing system, an additional amino acid sequence of an additional protein that is not included in the plurality of proteins;
identifying, by the computing system, a position of the additional amino sequence, the position corresponding to an initial amino acid included in the additional amino acid sequence;
analyzing the additional amino acid sequence, by the computing system and using the training data, with respect to a plurality of candidate amino acids to determine respective probabilities for individual candidate amino acids of the plurality of candidate amino acids being located at the position;
determining, by the computing system and based on the respective probabilities, an amount of loss of the initial amino acid being located at the position;
determining, by the computing system and based on the amount of loss, a candidate amino acid from among the plurality of candidate amino acids to replace the initial amino acid at the position; and
generating, by the computing system, a modified version of the additional amino acid sequence having the candidate amino acid located at the position.

2. The method of claim 1, wherein a value of a biophysical property of a variant protein corresponding to the modified version of the amino acid sequence is greater than an additional value of the biophysical property of the additional protein corresponding to the initial amino acid sequence.

3. The method of claim 1, wherein a measure of stability of a variant protein corresponding to the modified version of the amino acid sequence is greater than an additional measure of stability of the additional protein corresponding to the initial amino acid sequence.

4. The method of claim 1, comprising:
determining, by the computing system, that the amount of loss of the initial amino acid being located at the position is at least a threshold amount of loss; and
determining, by the computing system, that the initial amino acid is to be replaced based on the amount of loss of the initial amino acid being at least the threshold amount of loss.

5. The method of claim 1, comprising:
determining, by the computing system, that a first probability of the candidate amino acid being located at the position is greater than a second probability of the initial amino acid being located at the position; and
determining, by the computing system, that the initial amino acid is to be replaced based on the first probability being greater than the second probability.

6. The method of claim 1, comprising:
performing, by the computing system, a first training process using first additional training to produce a trained generating component of a generative adversarial network, the first additional training data including a first additional plurality of amino acid sequences of first additional proteins;
obtaining, by the computing system, a second additional training data that includes a second additional plurality of amino acid sequences of second additional proteins, the second additional proteins including a greater number of proteins having at least one of a structural feature or a biophysical property than the first additional plurality of first proteins included in the first additional training data;
performing, by the computing system and using the second additional training data, a second training process for a generative adversarial network that includes the trained generating component; and
producing, by the computing system, an additional trained generating component in relation to the second training process, the additional trained generating component generating at least a portion of the training data.

7. The method of claim 1, wherein a convolutional neural network is implemented to analyze the additional amino acid sequence and determine the respective probabilities, the convolutional neural network having a plurality of residual layers.

8. The method of claim 7, wherein:
the convolutional neural network includes a first residual component that provides at least a portion of the output of the first residual component to a second residual component;
the first residual component analyzes amino acid sequences in accordance with a first dilation rate; and
the second residual component analyzes amino acid sequences in accordance with a second dilation rate.

9. The method of claim 1, comprising:
determining, by the computing system, individual amounts of loss for a plurality of positions of the additional amino acid sequence;
determining, by the computing system, an aggregate amount of loss based on the individual amounts of loss; and
determining, by the computing system, a score for the additional amino acid sequence based on the aggregate amount of loss.

10. The method of claim 9, comprising:
determining, by the computing system, additional individual amounts of loss for an additional plurality of positions of the modified version of additional amino acid sequence;
determining, by the computing system, an additional aggregate amount of loss based on the additional individual amounts of loss; and
determining, by the computing system, an additional score for the modified version of the additional amino acid sequence.

11. The method of claim 10, wherein:
the score for the additional amino acid sequence is lower than the additional score for the modified version of the additional amino acid sequence; and
a first value of a biophysical property for the additional amino acid sequence is less than a second value of the biophysical property for the modified version of the additional amino acid sequence.

12. The method of claim 10, wherein:
the score for the additional amino acid sequence is lower than the additional score for the modified version of the additional amino acid sequence; and
the modified version of the additional amino acid sequence has a greater amount of homology with respect to amino acid sequences of proteins generated in accordance with one or more germline genomic regions than the additional amino acid sequence.

13. A system comprising:
one or more hardware processing units; and
one or more non-transitory memory devices storing computer-readable instructions that, when executed by the one or more hardware processing units, cause the system to perform operations comprising:
obtaining training data including a plurality of amino acid sequences, individual amino acid sequences of the plurality of amino acid sequences corresponding to an antibody fragment of a plurality of antibody fragments;
obtaining an additional amino acid sequence of an additional antibody component that is not included in the plurality of antibody components;
identifying a position of the additional amino sequence, the position corresponding to an initial amino acid included in the additional amino acid sequence;
analyzing the additional amino acid sequence, using the training data, with respect to a plurality of candidate amino acids to determine respective probabilities for individual candidate amino acids of the plurality of candidate amino acids being located at the position; and
determining, based on the respective probabilities, an amount of loss of the initial amino acid being located at the position.

14. The system of claim 13, wherein a convolutional neural network is implemented to analyze the additional amino acid sequence to determine respective probabilities for the individual candidate amino acid sequences and to determine the amount of loss of the initial amino acid being located at the position.

15. The system of claim 14, wherein the convolutional neural network includes a plurality of residual layers and a plurality of one-dimensional convolutional layers.

16. The system of claim 14, wherein the convolutional neural network implements one or more first models that analyze heavy chain variable regions of antibody sequences and one or more second models that analyze light chain variable regions of antibody sequences.

17. The system of claim 16, wherein the one or more second models include at least one second model to analyze kappa light chain variable regions of antibody sequences and at least one additional second model to analyze lambda light chain variable regions of antibody sequences.

18. The system of claim 16, wherein the one or more non-transitory memory devices store additional computer-readable instructions that, when executed by the one or more hardware processing units, cause the system to perform additional operations comprising:
generating, based on a first base sequence, a first amino acid sequence corresponding to a first variant sequence of a heavy chain variable region;
generating, based on a second base sequence, a second amino acid sequence corresponding to a second variant sequence of a light chain variable region; and
combining the first amino acid sequence and the second amino acid sequence to generate an antibody amino acid sequence that includes the heavy chain variable region and the light chain variable region.

19. A method comprising:
obtaining, by a computing system including one or more computing devices having one or more processors and memory, training data including a plurality of amino acid sequences, individual amino acid sequences of the plurality of amino acid sequences corresponding to an individual protein of a plurality of proteins;
obtaining, by the computing system, a base amino acid sequence of at least a portion of a first additional protein that is not included in the plurality of proteins;
obtaining, by the computing system, a grafting sequence of at least a portion of a second additional protein that is not included in the plurality of proteins;
determining, by the computing system and based on position modification data, a plurality of first positions of the base sequence that include first amino acids that are to be modified;
determining, by the computing system and based on the position modification data, a plurality of second positions of the grafting sequence that include second amino acids that are to replace the first amino acids;
generating, by the computing system, a combined amino acid sequence that includes a modified version of the base sequence with the first amino acids being replaced by the second amino acids;
analyzing the combined amino acid sequence, by the computing system and using the training data, with respect to a plurality of candidate amino acids to determine respective probabilities for individual candidate amino acids of the plurality of candidate amino acids being located at a position of the combined sequence;
determining, by the computing system and based on the respective probabilities, an amount of loss of an initial amino acid being located at the position.
determining, by the computing system and based on the amount of loss, a candidate amino acid from among the plurality of candidate amino acids to replace the initial amino acid at the position; and
generating, by the computing system, a modified version of the additional amino acid sequence having the candidate amino acid located at the position.

20. The method of claim 19, wherein the first additional protein is produced according to one or more human genomic regions; and the second additional protein is produced according to one or more non-human mammalian genomic regions.
